(19) United States
(12) Patent Application Publication
(10) Pub. No.: US 2025/0179465 A1
(43) Pub. Date: Jun. 5, 2025

(54) SITE-SPECIFIC ENCODING OF FLUORINATED PHENYLALANINE RESIDUES IN BACTERIAL AND MAMMALIAN CELLS

(71) Applicants: University of Iowa Research Foundation, Iowa City, IA (US); Oregon State University, Corvallis, OR (US)

(72) Inventors: Christopher Ahern, Iowa City, IA (US); Daniel Infield, Iowa City, IA (US); Richard Cooley, Corvallis, OR (US); Ryan Mehl, Corvallis, OR (US)

(73) Assignees: University of Iowa Research Foundation, Iowa City, IA (US); Oregon State University, Corvallis, OR (US)

(21) Appl. No.: 18/839,235

(22) PCT Filed: Feb. 16, 2023
(86) PCT No.: PCT/US2023/013254

§ 371 Date: Aug. 16, 2024

Related U.S. Application Data
(60) Provisional application No. 63/311,941, filed on Feb. 18, 2022.
(60) Provisional application No. 63/412,098, filed on Sep. 30, 2022.

(51) Int. Cl. C12N 9/00; C12N 15/70; C12N 15/85

(52) U.S. Cl. CPC C12N 9/93; C12N 15/70; C12N 15/85; C12Y 601/01026; C12N 2800/101; C12N 2800/107

(57) ABSTRACT

The present invention provides in certain embodiments comprising a pyrrolysine-based aminoacyl-tRNA synthetase and a tRNA to form a pyrrolysine-based aminoacyl-tRNA synthetase/tRNA pair. The present invention also provides novel pyrrolysine-based aminoacyl-tRNA synthetases, cells and compositions comprising pyrrolysine-based aminoacyl-tRNA synthetases. The present invention also provides methods of using these pyrrolysine-based aminoacyl-tRNA synthetases.

### RELATED APPLICATIONS

This application claims priority to U.S. Provisional Application No. 63/311,941 that was filed on Feb. 18, 2022 and U.S. Provisional Application No. 63/412,098 that was filed on Sep. 30, 2022. The entire content of the applications referenced above is hereby incorporated by reference herein.

### STATEMENT REGARDING FEDERALLY SPONSORED RESEARCH

This invention was made with government support under HL149184, NS104617, and GM131168 awarded by National Institutes of Health. The government has certain rights in the invention.

### BACKGROUND

The aromatic side-chains of phenylalanine, tyrosine, and tryptophan are crucial for protein function and pharmacology due to their hydrophobic and electrostatic contributions to catalytic centers and ligand-binding pockets. Few experimental approaches, however, can chemically assess the functional roles of aromatics in cellular environments. The accepted computational method for aromatic interrogation is via serial fluorination, such that the interaction energy of a purely electrostatic interaction will decrease linearly with each added fluorine. Experimental systems that use this approach, however, are limited to the Xenopus oocyte expression system, which is unsuitable for many human and/or soluble proteins. Accordingly, new approaches are needed to chemically assess the functional roles of aromatics in cellular environments. Also needed are fluorinated aromatic amino acids that confer supra-physiological stability to protein therapeutics.

### SUMMARY

The present invention provides in certain embodiments a composition comprising a pyrrolysine-based aminoacyl-tRNA synthetase and a tRNA to form a pyrrolysine-based aminoacyl-tRNA synthetase/tRNA pair.

The present invention provides in certain embodiments a method of site-specific encoding of a fluorinated phenylalanine amino acid in a cell comprising co-transfecting the cell with synthetase Phe_{X-B5} or Phe_{X-D6} and contacting the cell with 2,3,6F Phe.

In certain embodiments, the cell is a prokaryotic cell.

In certain embodiments, the prokaryotic cell is an E. coli cell.

In certain embodiments, the cell is a eukaryotic cell.

In certain embodiments, the eukaryotic cell is mammalian cell.

The present invention provides in certain embodiments a cell comprising an artificially evolved tRNA synthetase. The present invention provides in certain embodiments a cell comprising synthetase Phe_{X-B5}, Phe_{X-D6}, Phe_{X-A12}, Phe_{X-B6}, Phe_{X-C4}, Phe_{X-D7}, or Phe_{X-A11}. In certain embodiments, the cell is a prokaryotic cell. In certain embodiments, the cell is a eukaryotic cell.

The present invention provides in certain embodiments a cell free system comprising a pyrrolysine-based aminoacyl-tRNA synthetase and a tRNA to form a pyrrolysine-based aminoacyl-tRNA synthetase/tRNA pair.

The present invention provides in certain embodiments a Phex synthetase that is competent for site-specific encoding of fluorinated di-, tr-, tetra- and penta-fluoro phenylalanine analogs within protein in both bacterial and mammalian expression systems. As used herein, a “competent Phex synthetase” is one that is capable of catalyzing an aminoacylation reaction by covalently linking a fluorinated di-, tr-, tetra- and penta-fluoro phenylalanine with its cognate tRNA.

The present invention provides in certain embodiments a composition comprising synthetase Phe_{X-B5}, Phe_{X-D6}, Phe_{X-A12}, Phe_{X-B6}, Phe_{X-C4}, Phe_{X-D7}, or Phe_{X-A11}. In certain embodiments, the Pyrrolysine-based aminoacyl-tRNA synthetase is generated in E. coli. In certain embodiments, the Pyrrolysine-based aminoacyl-tRNA synthetase is generated in a mammalian cell. In certain embodiments, the mammalian cell is a human embryonic kidney (HEK) cell.

The present invention provides in certain embodiments a vector comprising a plasmid and a nucleic acid encoding synthetases Phe_{X-D6} or Phe_{X-B5}.

In certain embodiments, the plasmid is pAcBac1.

The present invention provides in certain embodiments a method of expressing sfGFP_N150TAG_HIS in a cell by contacting the cell with Phe_{X-D6} and Phe_{X-B5}.

In certain embodiments, the cell is a prokaryotic cell.

In certain embodiments, the prokaryotic cell is an E. coli cell.

In certain embodiments, the cell is a eukaryotic cell.

In certain embodiments, the eukaryotic cell is mammalian cell.

### BRIEF DESCRIPTION OF DRAWINGS

FIGS. 1A-1B. FIG. 1A. Illustrations of types of pi interactions in which Phenylalanine can participate. FIG. 1B. Electrostatic views of benzine (as a structural surrogate for Phenylalanine) and fluorinated analogs.

FIGS. 2A-2D. Strategy and identification of synthetases. FIG. 2A. Chemical structure of para-methyl tetra-fluoro Phe, which was used for screening in a PylRs library in E. coli. FIG. 2B. Heatmap of synthetases hits and relative GFP N150TAG rescue with para-methyl tetra-fluoro Phe. Rounds 1 and 2 were independent screens of the library with para-methyl tetra-fluoro Phe. FIG. 2C. Table of active site and off-target mutations borne by the top performing identified synthetases. FIG. 2D. Potency (UP50) curves for the synthetases detailed in FIG. 2C.

FIGS. 3A-3C. Permissivity of the identified synthetases. FIG. 3A. Chemical structures of the fluorinated phenylalanine residues used in the screen. FIG. 3B. Green fluorescence from the rescue of sfGFP-N150TAG in E. coli using 1 mM of each ncAA after 24-hour incubation. The bars in the graphs from left to right are the following:
-ncAA
2-monofluoro-Phe
4-monofluoro-Phe
2,4-difluoro-Phe
2,5-difluoro-Phe
2,6-difluoro-Phe
3,5-difluoro-Phe
3,4,5-trifluoro-Phe
2,3,6-trifluoro-Phe
2,3,4,5-tetrafluoro-Phe
2,3,5,6-tetrafluoro
4-Me-2,3,5,6-tetrafluoro-Phe
Pentafluoro-Phe
   FIG. 3C. Effects of fluorination on interactions energies with cations.

FIGS. 4A-4B. FIG. 4A. ESI mass spectrometry confirmation of site-specific encoding of florinated Phe residues within superfolder GFP in E. coli. sfGFP was purified via His tag from 50 mL growths. Table at top right: mass differences of sfGFP N150TAG from WT GFP conform to predication for the molecular mass of each fluoro Phe species. In some condition for B5 synthetase, a small fraction of mass consistent with Phe incorporation was observed (asterisks). Mass spectrometry was not pursued for the combination of 2F Phe and D6 due to low expression in the screen. FIG. 4B. Protein sequence of E. coli expression sfGFP_N150TAG_HIS variant used for expression and purification in this study (SEQ ID NO: 15).

FIGS. 5A-5C. Function of Pheₓ synthetases in HEKT cells. FIG. 5A. Micrographs of HEKT cells co-transfected with GFP_n150TAG and Phe_{X-D6} in the presence or absence of the given amino acid. GFP (top) and brightfield (bottom). In all cases, images taken approximately 24 hours after transfection and unnatural amino acid used at 2 mM concentration. FIG. 5B. Lysate GFP fluorescence for a range of fluorinated phenylalanine residues. The bars in the graphs from left to right are the following:
-ncAA
2-monofiuoro-Phe
2.4-difluoro-Phe
2,5-difluoro-Phe
2.6-difworo-Pt
3,4,5-trifluoro-Phe
2,3,6-trifiuoro-Phe
2,3,4,5-tetrafiuoro-Phe
2,3,5,6-tetrafiuoro
Pentafiuoro-Phe
   FIG. 5C. Protein sequence of HEK cell expression sfGFP_N150TAG_V5_HIS variant used for expression and purification in this study (SEQ ID NO:16).

FIGS. 6A-6B. FIG. 6A. ESI mass spec confirmation of encoding of fluorinated Phe analogs within sfGFP expressed in HEKT cells. ncAAs were added a 2 mM concentration and cells were harvested approximately 36 hours post transfection. sfGFP-WT-V5-HIS or N150_TAG-V5-HIS was purified via HIS tag. Table (at top right) report mass differences of sfGFP N150TAG from WT GFP, which conform to prediction for the molecular mass of each fluoro Phe species. Masses confirmed high fidelity encoding of di, tri, tetra, and pentafluoro phenylalanine. Yield of sfGFP N150 (PENTA) via Phe_{X-D6} was ˜30 μg/g cells. FIG. 6B. GFP lysate values from HEKT cells co-transfected with sfGFP150TAG and either Phe_{X-D6} or Phe_{X-B5} and harvested approximately 24 hours post transfection. Error bars are±STD. In the left graph of FIG. 6B, starting from the top, the first curve is pentafluoro-Phe; the second curve is 2,3,5,6-tetrafluoro; the third curve is 2,3,4,5-tetrafluoro-Phe; the fourth curve is 2,3,6-trifluoro-Phe; the fifth curve is 2,6-difluoro-Phe; the six curve is 3,4,5-trifluoro-Phe; and the last curve (at the bottom) is 2,4-difluoro-Phe.

FIGS. 7A-7H. Site-specific encoding of tri-fluoro-Phe within two large, human ion channels. FIG. 7A Western blot of CFTR using C terminal antibody (ab596) of WT CFTR or F508TAG-CFTR co-transfected with Phe_{x-D6} in the presence or absence of 2 mM 2,3,6 F Phe. WT lysates are loaded at 1:10 of TAG conditions. FIG. 7B. Western blot of Nav 1.5 using C terminal antibody (D9J7S) of WT hNaᵥ 1.5 or F1486TAG-hNaᵥ 1.5 co-transfected with Phe_{x-D6} in the presence or absence of 2 mM 2,3,6 F Phe. FIG. 7C. Quantification (densitometry) of signal from 60 μg of loaded lysate. FIG. 7D. Mass spectra of tryptic fragment from expressed and purified hNav 1.5 F1486(2,3,6 F Phe) via D6. FIG. 7E. Example current families for whole (HEK) cells expressing WT or mutant channels. Cells held at −140 mV and activated via depolarization in 5 mV increments. FIG. 7F. Activation (G/V) curves for WT and mutant channels. FIG. 7G. Steady state inactivation curves for WT and mutant channels. FIG. 7H. Gating Parameters for hNaᵥ 1.5 variants. Bolded=p value of 0.0014 compared to WT. P>0.24 for all other comparisons.

### DETAILED DESCRIPTION

The amino acid phenylalanine is crucial for protein function, stability, and pharmacology due to hydrophobic and electrostatic contributions from its aromatic side chain. Fluorination (hydrogen-to-fluorine substitution) on the aryl ring of phenylalanine allows for precise atomic tuning of hydrophobic and electrostatic forces without substantially changing the overall shape of the amino acid.

The inventors invented a family of novel, recombinant amino-acyl tRNA synthetase enzymes that are useful in making recombinant proteins that include at least one fluorinated phenylalanine amino acid. Thus, in addition to the novel recombinant animo-acyl tRNA enzymes, the present invention provides for recombinant proteins that incorporate a fluorinated phenylalanine amino acid. In some cases, the fluorinated phenylalanine containing recombinant protein may be a therapeutic protein.

The recombinant amino-acyl tRNA synthetase enzymes may be co-expressed in a cell with a corresponding tRNA to enable the robust, site-specific, scalable encoding of multiple different types of fluorinated phenylalanine amino acids in recombinant proteins expressed in mammalian cell lines and bacteria. In some examples, the recombinant amino-acyl tRNA synthetase enzyme is a pyrrolysine tRNA from archaea. These novel enzymes thus enable the advantageous qualities of site-specific fluorination, including enhanced protein stability, resistance to proteolysis, and manipulation of ligand/receptor interactions, to be applied to protein engineering in industrial and biomedical applications.

The recombinant amino-acyl tRNA synthetase enzyme and a corresponding tRNA may also be used in a cell free system to produce recombinant proteins that incorporate a fluorinated phenylalanine amino acid.

### Pyrrolysine-Based Aminoacyl-tRNA Synthetases

The present invention provides in certain embodiments novel pyrrolysine-based aminoacyl-tRNA synthetases. The present invention provides in certain embodiments, a Pheₓ synthetase that is competent for site-specific encoding of fluorinated di-, tr-, tetra- and penta-fluoro phenylalanine analogs within protein in both bacterial and mammalian expression systems.

The present invention provides in certain embodiments comprises synthetase Phe_{X-B5}, Phe_{X-D6}, Phe_{X-A12}, Phe_{X-B6}, Phe_{X-C4}, Phe_{X-D7}, or Phe_{X-A11}. In certain embodiments, the synthetase is Phe_{X-B5}. In certain embodiments, the synthetase is Phe_{X-D6}. In certain embodiments, the synthetase is Phe_{X-A12}. In certain embodiments, the synthetase is Phe_{X-B6}. In certain embodiments, the synthetase is Phe_{X-C4}. In certain embodiments, the synthetase is Phe_{X-D7}. In certain embodiments, the synthetase is Phe_{X-A11}. In certain embodiments, the Pyrrolysine-based aminoacyl-tRNA synthetase is generated in E. coli. In certain embodiments, the Pyrrolysine-based aminoacyl-tRNA synthetase is generated in a mammalian cell. In certain embodiments, the mammalian cell is a human embryonic kidney (HEK) cell. In certain embodiments, the pyrrolysine-based aminoacyl-tRNA synthetase has the sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO: 11, SEQ ID NO: 12, SEQ ID NO: 13, or SEQ ID NO: 14.

The present invention provides in certain embodiments, a Pheₓ synthetase that is a “competen” t Pheₓ synthetase, in that it is capable of catalyzing an aminoacylation reaction by covalently linking a fluorinated di-, tr-, tetra- and penta-fluoro phenylalanine with its cognate tRNA in both bacterial and mammalian expression systems.

In certain embodiments, the synthetase is a Pyrrolysine-based aminoacyl-tRNA synthetase generated in E. coli. In certain embodiments, the the Pyrrolysine-based aminoacyl-tRNA synthetase is Phe_{X-B5} (SEQ ID NO:3), Phe_{X-D6} (SEQ ID NO:1), Phe_{X-A12} (SEQ ID NO:5), Phe_{X-B6} (SEQ ID NO:7), Phe_{X-C4} (SEQ ID NO:9), Phe_{X-D7} (SEQ ID NO:11), or Phe_{X-A11} (SEQ ID NO:13).

In certain embodiments, the synthetase is a Pyrrolysine-based aminoacyl-tRNA synthetase generated in a mammalian cell. In certain embodiments, the the Pyrrolysine-based aminoacyl-tRNA synthetase is Phe_{X-B5} (SEQ ID NO:4), Phe_{X-D6} (SEQ ID NO:2), Phe_{X-A12} (SEQ ID NO:6), Phe_{X-B6} (SEQ ID NO:8), Phe_{X-C4} (SEQ ID NO:10), Phe_{X-D7} (SEQ ID NO:12), or Phe_{X-A11} (SEQ ID NO:14).

### Pyrrolysine-Based Aminoacyl-tRNA Synthetase/tRNA Pairs

The present invention provides in certain embodiments, a novel pyrrolysine-based aminoacyl-tRNA synthetase (RS) paired with a tRNA to form a pyrrolysine-based aminoacyl-tRNA synthetase/tRNA pair (RS/tRNA pair). In certain embodiments the RS and the tRNA are the following:

RS (E. coli compatible)

Phe_{X-B5} (SEQ ID NO: 3)
Phe_{X-D6} (SEQ ID NO: 1)
Phe_{X-A12} (SEQ ID NO: 5)
Phe_{X-B6} (SEQ ID NO: 7)
Phe_{X-C4} (SEQ ID NO: 9)
Phe_{X-D7} (SEQ ID NO: 11)
Phe_{X-A11} (SEQ ID NO: 13)

RS (mammalian compatible)

Phe_{X-B5} (SEQ ID NO: 4)
Phe_{X-D6} (SEQ ID NO: 2)
Phe_{X-A12} (SEQ ID NO: 6)
Phe_{X-B6} (SEQ ID NO: 8)
Phe_{X-C4} (SEQ ID NO: 10)
Phe_{X-D7} (SEQ ID NO: 12)
Phe_{X-A11} (SEQ ID NO: 14)

All of the RS enzymes are paired with a naturally occurring pyrollysine tRNA (PylT) from Methanogenic archaea:

(SEQ ID NO: 17)
GGAAACCUGAUCAUGUAGAUCGAACGGACUCUAAAUCCGUUCAGCCGG
GUUAGAUUCCCGGGGUUUCCGCCA.

Note that the relevant synthetase family (PylRs) has been previously shown in literature to work with many variants of PylT (multiple species, well as versions where the anticodon has been altered to recognize other stop codons (TGA, TAA, in addition to the natural TAG). In expression plasmids for this system, this sequence exists as DNA, which is transcribed to tRNA in the cell. The plasmid sequences often lack the last-CCA, which is added endogenously by the cell.

(SEQ ID NO: 18)
GGAAACCUGAUCAUGUAGAUCGAACGGACUCUAAAUCCGUUCAGCCGG
GUUAGAUUCCCGGGGUUUCCGCCA.

### Compositions

The present invention provides in certain embodiments a cell comprising an artificially evolved tRNA synthetase. “Artificially evolved tRNA synthetases” are synthetases that are identified out of a man-made library of synthetases with randomized active sites. The present invention provides in certain embodiments a cell comprising synthetase Phe_{X-B5} (SEQ ID NO:3), Phe_{X-D6} (SEQ ID NO:1), Phe_{X-A12} (SEQ ID NO:5), Phe_{X-B6} (SEQ ID NO:7), Phe_{X-C4} (SEQ ID NO:9), Phe_{X-D7} (SEQ ID NO:11), Phe_{X-A11} (SEQ ID NO: 13), Phe_{X-B5} (SEQ ID NO:4), Phe_{X-D6} (SEQ ID NO:2), Phe_{X-A12} (SEQ ID NO:6), Phe_{X-B6} (SEQ ID NO:8), Phe_{X-C4} (SEQ ID NO:10), Phe_{X-D7} (SEQ ID NO:12), or Phe_{X-A11} (SEQ ID NO: 12). In certain embodiments, the cell is a prokaryotic cell. The present invention provides in certain embodiments a cell comprising synthetase Phe_{X-B5} (SEQ ID NO:4), Phe_{X-D6} (SEQ ID NO:2), Phe_{X-A12} (SEQ ID NO:6), Phe_{X-B6} (SEQ ID NO:8), Phe_{X-C4} (SEQ ID NO: 10), Phe_{X-D7} (SEQ ID NO:12), or Phe_{X-A11} (SEQ ID NO:12). In certain embodiments, the cell is a eukaryotic cell.

The present invention provides in certain embodiments, a composition containing a pyrrolysine-based aminoacyl-tRNA synthetase and a tRNA to form a pyrrolysine-based aminoacyl-tRNA synthetase/tRNA pair, and further containing a non-canonical amino acid (ncAA). The enzymes enable the installation of a wide variety of fluorinated amino acids, including 2-mono fluoro Phenylalanine, 2,6 di-fluoro Phenylalanine, 2,3,6 tri-fluoro Phenylalanine, 2,3,5,6 tetra-fluoro Phenylalanine, 2,3,4,5 tetra-fluoro Phenylalanine, 2,3,4,5,6 penta-fluoro Phenylalanine, para-methyl-tetrafluoro Phenylalanine, para-methyl-2-mono fluoro Phenylalanine, and para-methyl-2,3-di-fluoro Phenylalanine.

The present invention provides in certain embodiments, a composition comprising a cell free protein translation system comprising a pyrrolysine-based aminoacyl-tRNA synthetase and a tRNA to form a pyrrolysine-based aminoacyl-tRNA synthetase/tRNA pair.

### Vectors

The present invention provides in certain embodiments, a vector comprising a plasmid and a nucleic acid encoding a synthetase. In certain embodiments the synthetase is a novel pyrrolysine-based aminoacyl-tRNA synthetase. The present invention provides in certain embodiments, a Pheₓ synthetase that is competent for site-specific encoding of fluorinated di-, tr-, tetra- and penta-fluoro phenylalanine analogs within protein in both bacterial and mammalian expression systems.

The present invention provides in certain embodiments wherein the vector comprises synthetase Phe_{X-B5}, Phe_{X-D6}, Phe_{X-A12}, Phe_{X-B6}, Phe_{X-C4}, Phe_{X-D7}, or Phe_{X-A11}. In certain embodiments, the synthetase is Phe_{X-B5}. In certain embodiments, the synthetase is Phe_{X-D6}. In certain embodiments, the synthetase is Phe_{X-A12}. In certain embodiments, the synthetase is Phe_{X-B6}. In certain embodiments, the synthetase is Phe_{X-C4}. In certain embodiments, the synthetase is Phe_{X-D7}. In certain embodiments, the synthetase is Phe_{X-A11}. In certain embodiments, the Pyrrolysine-based aminoacyl-tRNA synthetase is generated in E. coli. In certain embodiments, the Pyrrolysine-based aminoacyl-tRNA synthetase is generated in a mammalian cell. In certain embodiments, the mammalian cell is a human embryonic kidney (HEK) cell. In certain embodiments, the pyrrolysine-based aminoacyl-tRNA synthetase has the sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO: 11, SEQ ID NO: 12, SEQ ID NO: 13, or SEQ ID NO: 14.

The present invention provides in certain embodiments, a vector encoding a Pheₓ synthetase that is a competent Phex synthetase that is capable of catalyzing an aminoacylation reaction by covalently linking a fluorinated di-, tr-, tetra- and penta-fluoro phenylalanine with its cognate tRNA.

In certain embodiments, the plasmid is pAcBac1.

### Methods of Use

Recombinant proteins are produced for many therapeutic and industrial applications. Site specific fluorination of constituent amino acids is known to increase stability and confer resistance to proteolytic digestion. This system enables the design and scalable production of optimized, fluorinated proteins for these applications. A second, distinct commercial use includes the use of the system for the study of ligand-protein interactions.

The present invention provides in certain embodiments, a method of site-specific encoding of a fluorinated phenylalanine amino acid in a cell comprising co-transfecting the cell with synthetase Phe_{X-B5} (SEQ ID NO:3), Phe_{X-D6} (SEQ ID NO:1), Phe_{X-A12} (SEQ ID NO: 5), Phe_{X-B6} (SEQ ID NO:7), Phe_{X-C4} (SEQ ID NO:9), Phe_{X-D7} (SEQ ID NO:11), Phe_{X-A11} (SEQ ID NO:13), Phe_{X-B5} (SEQ ID NO:4), Phe_{X-D6} (SEQ ID NO:2), Phe_{X-A12} (SEQ ID NO:6), Phe_{X-B6} (SEQ ID NO:8), Phe_{X-C4} (SEQ ID NO:10), Phe_{X-D7} (SEQ ID NO: 12), or Phe_{X-A11} (SEQ ID NO:12) and contacting the cell with 2,3,6F Phe. In certain embodiments, the synthetase is Phe_{X-B5} (SEQ ID NO:3), Phe_{X-D6} (SEQ ID NO:1), Phe_{X-B5} (SEQ ID NO:4), or Phe_{X-D6} (SEQ ID NO:2).

In certain embodiments, the the cell is a prokaryotic cell, and the synthetase is Phe_{X-B5} (SEQ ID NO:3), Phe_{X-D6} (SEQ ID NO:1), Phe_{X-A12} (SEQ ID NO:5), Phe_{X-B6} (SEQ ID NO:7), Phe_{X-C4} (SEQ ID NO:9), Phe_{X-D7} (SEQ ID NO:11), or Phe_{X-A11} (SEQ ID NO:13). In certain embodiments, the prokaryotic cell is an E. coli cell.

In certain embodiments, the the cell is a eukaryotic cell, and the synthetase is Phe_{X-B5} (SEQ ID NO:4), Phe_{X-D6} (SEQ ID NO:2), Phe_{X-A12} (SEQ ID NO:6), Phe_{X-B6} (SEQ ID NO:8), Phe_{X-C4} (SEQ ID NO:10), Phe_{X-D7} (SEQ ID NO:12), or Phe_{X-A11} (SEQ ID NO:12). In certain embodiments, the eukaryotic cell is a mammalian cell.

In certain embodiments, the present invention provides a method of expressing sfGFP_N150TAG HIS in a cell by contacting the cell with Phe_{X-B5} (SEQ ID NO:3), Phe_{X-D6} (SEQ ID NO:1), Phe_{X-A12} (SEQ ID NO:5), Phe_{X-B6} (SEQ ID NO:7), Phe_{X-C4} (SEQ ID NO:9), Phe_{X-D7} (SEQ ID NO:11), or Phe_{X-A11} (SEQ ID NO:13).

In certain embodiments, the present invention provides a method of expressing sfGFP_N150TAG_HIS in a cell by contacting the cell with Phe_{X-B5} (SEQ ID NO:4), Phe_{X-D6} (SEQ ID NO:2), Phe_{X-A12} (SEQ ID NO:6), Phe_{X-B6} (SEQ ID NO:8), Phe_{X-C4} (SEQ ID NO:10), Phe_{X-D7} (SEQ ID NO:12), and Phe_{X-A11} (SEQ ID NO:14).

In certain embodiments, the present invention provides a method of expressing sfGFP_N150TAG_HIS in a cell by contacting the cell with Phe_{X-D6} or Phe_{X-B5}. In certain embodiments, the cell is a prokaryotic cell. In certain embodiments, the prokaryotic cell is an E. coli cell. In certain embodiments, the cell is a eukaryotic cell. In certain embodiments, the eukaryotic cell is mammalian cell.

### Pharmaceutical Compositions

The present invention also provides, in certain embodiments, a process for producing a pharmaceutical composition comprising combining a composition as described herein with a pharmaceutically acceptable carrier. In certain embodiments, the composition is a cell comprising an artificially evolved tRNA synthetase. In certain embodiments, the composition is a cell comprising synthetase Phe_{X-B5}, Phe_{X-D6}, Phe_{X-A12}, Phe_{X-B6}, Phe_{X-C4}, Phe_{X-D7}, or Phe_{X-A11}. In certain embodiments, the cell is a prokaryotic cell. In certain embodiments, the cell is a eukaryotic cell.

In certain embodiments, the composition is a pyrrolysine-based aminoacyl-tRNA synthetase and a tRNA to form a pyrrolysine-based aminoacyl-tRNA synthetase/tRNA pair, and further containing a non-canonical amino acid (ncAA). The enzymes enable the installation of a wide variety of fluorinated amino acids, including 2-mono fluoro Phenylalanine, 2,6 di-fluoro Phenylalanine, 2,3,6 tri-fluoro Phenylalanine, 2,3,5,6 tetra-fluoro Phenylalanine, 2,3,4,5 tetra-fluoro Phenylalanine, 2,3,4,5,6 penta-fluoro Phenylalanine, para-methyl-tetrafluoro Phenylalanine, para-methyl-2-mono fluoro Phenylalanine, and para-methyl-2,3-di-fluoro Phenylalanine.

The present invention also provides, in certain embodiments, composition comprising a cell free protein translation system comprising a pyrrolysine-based aminoacyl-tRNA synthetase and a tRNA to form a pyrrolysine-based aminoacyl-tRNA synthetase/tRNA pair. The present invention provides, in certain embodiments, a pharmaceutical composition which comprises a pharmaceutically acceptable carrier or diluent and, as an active ingredient, a conjugate or composition as described herein. In certain embodiments, the pharmaceutical composition is formulated for oral administration or injection.

The present invention also provides, in certain embodiments, a composition as described herein for use in a method of treatment of a human or animal body by therapy.

The present invention also provides, in certain embodiments, the use of a composition as described herein in the manufacture of a medicament for treating a disease or disorder.

### Nucleic Acids, Expression Cassettes and Vectors

The present invention further provides nucleic acid sequences that encode synthetases. The nucleic acids encoding the synthetase can be produced using the methods well known in the art (see, e.g., Sambrook and Russell, 2001).

Accordingly, in certain embodiments of the invention provide a nucleic acid molecule encoding a synthetase as described herein.

Certain embodiments of the invention provide an expression cassette comprising a nucleic acid molecule described herein. In certain embodiments, the expression cassette described herein further comprises a promoter, such as a regulatable promoter or a constitutive promoter. Examples of suitable promoters include a CMV, RSV, pol II or pol III promoter. The expression cassette may further contain a polyadenylation signal (such as a synthetic minimal polyadenylation signal) and/or a marker gene.

Certain embodiments of the invention provide a viral vector comprising an expression cassette described herein. Examples of appropriate vectors include adenoviral, lentiviral, adeno-associated viral (AAV), poliovirus, HSV, or murine Maloney-based viral vectors. In certain embodiments, the vector is an adenovirus (Ad).

Certain embodiments of the invention provide a plasmid vector comprising an expression cassette described herein.

The present invention provides cells (such as a mammalian cell) containing the expression cassette or vectors described above. The present invention also provides a non-human mammal containing the expression cassette or vectors described above.

### Definitions

“Bound” refers to binding or attachment that may be covalent, e.g., by chemically coupling, or non-covalent, e.g., ionic interactions, hydrophobic interactions, hydrogen bonds. Covalent bonds can be, for example, ester, ether, phosphoester, amide, peptide, imide, carbon-sulfur bonds, carbon-phosphorus bonds, and the like. The term “bound” includes terms such as “linked,” “conjugated,” “coupled,” “fused” and “attached.”

The invention encompasses isolated or substantially purified protein (or peptide) compositions. In the context of the present invention, an “isolated” or “purified” polypeptide is a polypeptide that exists apart from its native environment and is therefore not a product of nature. A polypeptide may exist in a purified form or may exist in a non-native environment such as, for example, a transgenic host cell. For example, an “isolated” or “purified” protein, or biologically active portion thereof, is substantially free of other cellular material, or culture medium when produced by recombinant techniques, or substantially free of chemical precursors or other chemicals when chemically synthesized. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed proteins or partial-length proteins encoded thereby are also encompassed by the present invention. By “fragment” or “portion” is meant a full length or less than full length of the amino acid sequence of, a polypeptide or protein.

“Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have in at least one embodiment 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Wild-type” refers to the normal gene, or organism found in nature without any known mutation.

“Operably-linked” refers to the association of molecules so that the function of one is affected by the other. For example, operably-linked nucleic acids refers to the association of nucleic acid sequences on single nucleic acid fragment so that the function of one is affected by the other, e.g., an arrangement of elements wherein the components so described are configured so as to perform their usual function. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. Control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

“Operably-linked” also refers to the association of proteins, such that a linked protein still functions as intended even when linked to another moiety. For example, if a is operably linked to tRNA, the resulting linked protein still retains the function of the synthetase.

The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to a reference sequence over a specified comparison window. Optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may results form, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. Conservative substitutions, such as exchanging one amino acid with another having similar properties, are preferred.

Thus, the polypeptides of the invention encompass naturally occurring proteins as well as variations and modified forms thereof. Such variants will continue to possess the desired activity. The deletions, insertions, and substitutions of the polypeptide sequence encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). In addition, individual substitutions, deletions or additions which alter, add or delete a single amino acid or a small percentage of amino acids in an encoded sequence are also “conservatively modified variations.”

As used herein, the term “nucleic acid” and “polynucleotide” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

A “nucleic acid fragment” is a portion of a given nucleic acid molecule.

Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA.

The term “gene” is used broadly to refer to any segment of nucleic acid associated with a biological function. Genes include coding sequences and/or the regulatory sequences required for their expression. For example, gene refers to a nucleic acid fragment that expresses mRNA, functional RNA, or a specific protein, including its regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. In addition, a “gene” or a “recombinant gene” refers to a nucleic acid molecule comprising an open reading frame and including at least one exon and (optionally) an intron sequence. The term “intron” refers to a DNA sequence present in a given gene which is not translated into protein and is generally found between exons.

A “vector” is defined to include, inter alia, any viral vector, as well as any plasmid, cosmid, phage or binary vector in double or single stranded linear or circular form that may or may not be self transmissible or mobilizable, and that can transform prokaryotic or eukaryotic host either by integration into the cellular genome or exist extrachromosomally (e.g., autonomous replicating plasmid with an origin of replication).

The term “transformation” refers to the transfer of a nucleic acid fragment into the genome of a host cell, resulting in genetically stable inheritance. A “host cell” is a cell that has been transformed, or is capable of transformation, by an exogenous nucleic acid molecule. Host cells containing the transformed nucleic acid fragments are referred to as “transgenic” cells.

“Transformed,” “transduced,” “transgenic” and “recombinant” refer to a host cell into which a heterologous nucleic acid molecule has been introduced. As used herein the term “transfection” refers to the delivery of DNA into eukaryotic (e.g., mammalian) cells. The term “transformation” is used herein to refer to delivery of DNA into prokaryotic (e.g., E. coli) cells. The term “transduction” is used herein to refer to infecting cells with viral particles. The nucleic acid molecule can be stably integrated into the genome generally known in the art. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

“Genetically altered cells” denotes cells which have been modified by the introduction of recombinant or heterologous nucleic acids (e.g., one or more DNA constructs or their RNA counterparts) and further includes the progeny of such cells which retain part or all of such genetic modification.

As used herein, the term “derived” or “directed to” with respect to a nucleotide molecule means that the molecule has complementary sequence identity to a particular molecule of interest.

“Expression cassette” as used herein means a nucleic acid sequence capable of directing expression of a particular nucleotide sequence in an appropriate host cell, which may include a promoter operably linked to the nucleotide sequence of interest that may be operably linked to termination signals. The coding region usually codes for a functional protein of interest, for example a synthetase. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of an regulatable promoter that initiates transcription only when the host cell is exposed to some particular stimulus. In the case of a multicellular organism, the promoter can also be specific to a particular tissue or organ or stage of development.

Such expression cassettes can include a transcriptional initiation region linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

The term “RNA transcript” or “transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” are nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, regulatable promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency.

“3′ non-coding sequence” refers to nucleotide sequences located 3′ (downstream) to a coding sequence and may include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

“Promoter” refers to a nucleotide sequence, usually upstream (5′) to its coding sequence, which directs and/or controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions. Examples of promoters that may be used in the present invention include the mouse U6 RNA promoters, synthetic human HIRNA promoters, SV40, CMV, RSV, RNA polymerase II and RNA polymerase III promoters.

“Constitutive expression” refers to expression using a constitutive or regulated promoter. “Conditional” and “regulated expression” refer to expression controlled by a regulated promoter.

“Expression” refers to the transcription and/or translation of an endogenous gene, heterologous gene or nucleic acid segment, or a transgene in cells. Expression may also refer to the production of protein.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

The following terms are used to describe the sequence relationships between two or more nucleic acids or polynucleotides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm.

Computer implementations of these mathematical algorithms can be utilized for comparison of sequences to determine sequence identity. Such implementations include, but are not limited to: CLUSTAL in the PC/Gene program (available from Intelligenetics, Mountain View, California); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wisconsin, USA). Alignments using these programs can be performed using the default parameters.

Software for performing BLAST analyses is publicly available through the National Center for Biotechnology Information (see the World Wide Web at ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. Tis referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, less than about 0.01, or even less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When using BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the World Wide Web at ncbi.n1m.nih.gov. Alignment may also be performed manually by visual inspection.

For purposes of the present invention, comparison of nucleotide sequences for determination of percent sequence identity to the sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by a BLAST program.

(c) As used herein, “sequence identity” or “identity” in the context of two nucleic acid sequences makes reference to a specified percentage of residues in the two sequences that are the same when aligned for maximum correspondence over a specified comparison window, as measured by sequence comparison algorithms or by visual inspection. When percentage of sequence identity is used in reference to proteins, it is recognized that residue positions that are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, or 79%; at least 80%, 81%, 82%, 83%, 84%, 85%, 86%, 87%, 88%, or 89%; at least 90%, 91%, 92%, 93%, or 94%; or even at least 95%, 96%, 97%, 98%, or 99% sequence identity, compared to a reference sequence using one of the alignment programs described using standard parameters.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code.

(e)(ii) For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The Tₘ is the temperature (under defined ionic strength and pH) at which 50% of the target sequence hybridizes to a perfectly matched nucleic acid. Specificity is typically the function of post-hybridization washes, the critical factors being the ionic strength and temperature of the final wash solution. For DNA-DNA hybrids, the Tₘ can be approximated from the equation of Meinkoth and Wahl:

Tₘ 81.5° C.+16.6 (log M)+0.41 (% GC)−0.61 (% form)−500/L
where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. Generally, highly stringent hybridization and wash conditions are selected to be about 5° C. lower than the thermal melting point (Tₘ) for the specific sequence at a defined ionic strength and pH.

An example of highly stringent wash conditions is 0.15 M NaCl at 72° C. for about 15 minutes. An example of stringent wash conditions is a 0.2× SSC wash at 65° C. for 15 minutes. Often, a high stringency wash is preceded by a low stringency wash to remove background probe signal. An example medium stringency wash for a duplex of, e.g., more than 100 nucleotides, is 1× SSC at 45° C. for 15 minutes. An example low stringency wash for a duplex of, e.g., more than 100 nucleotides, is 4-6× SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1× SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1× to 2× SSC (20× SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1× SSC at 55 to 60° C.

As discussed above, the terms “isolated and/or purified” in terms of a nucleic acid refer to in vitro isolation of a nucleic acid, e.g., a DNA or RNA molecule from its natural cellular environment, and from association with other components of the cell, such as nucleic acid or polypeptide, so that it can be sequenced, replicated, and/or expressed. For example, “isolated nucleic acid” may be a DNA molecule that is complementary or hybridizes to a sequence in a gene of interest and remains stably bound under stringent conditions (as defined by methods well known in the art). Thus, the RNA or DNA is “isolated” in that it is free from at least one contaminating nucleic acid with which it is normally associated in the natural source of the RNA or DNA and in one embodiment of the invention is substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell, e.g., in a vector or plasmid.

As used herein, the term “recombinant nucleic acid,” e.g., “recombinant DNA sequence or segment” refers to a nucleic acid, e.g., to DNA, that has been derived or isolated from any appropriate cellular source, that may be subsequently chemically altered in vitro, so that its sequence is not naturally occurring, or corresponds to naturally occurring sequences that are not positioned as they would be positioned in a genome that has not been transformed with exogenous DNA. An example of preselected DNA “derived” from a source would be a DNA sequence that is identified as a useful fragment within a given organism, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

Thus, recovery or isolation of a given fragment of DNA from a restriction digest can employ separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. Therefore, “recombinant DNA” includes completely synthetic DNA sequences, semi-synthetic DNA sequences, DNA sequences isolated from biological sources, and DNA sequences derived from RNA, as well as mixtures thereof.

Nucleic acid molecules having base substitutions (i.e., variants) are prepared by a variety of methods known in the art. These methods include, but are not limited to, isolation from a natural source (in the case of naturally occurring sequence variants) or preparation by oligonucleotide-mediated (or site-directed) mutagenesis, PCR mutagenesis, and cassette mutagenesis of an earlier prepared variant or a non-variant version of the nucleic acid molecule.

As used herein, the term “therapeutic agent” or “therapeutic complex” refers to any agent or material that has a beneficial effect on the mammalian recipient. Thus, “therapeutic agent” embraces both therapeutic and prophylactic molecules having nucleic acid or protein components.

“Treating” as used herein refers to ameliorating at least one symptom of, curing and/or preventing the development of a given disease or condition.

### Formulations and Methods of Administration

The compositions of the invention may be formulated as pharmaceutical compositions and administered to a mammalian host, such as a human patient, in a variety of forms adapted to the chosen route of administration, i.e., orally, intranasally, intradermally or parenterally, by intravenous, intramuscular, topical or subcutaneous routes. Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like;

a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions. For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Additional ingredients such as fragrances or antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions that can be used to deliver the compounds of the present invention to the skin are known to the art.

Useful dosages of the compounds of the present invention can be determined by comparing their in vitro activity, and in vivo activity in animal models. Methods for the extrapolation of effective dosages in mice, and other animals, to humans are known to the art.

Generally, the concentration of the compound(s) of the present invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

In certain embodiments, the vaccine of the present invention reduces the size of the tumor in the subject by at least about 10%-100% (volume of tumor).

### Nucleic Acid Molecules of the Invention

The terms “isolated and/or purified” refer to in vitro isolation of a nucleic acid, e.g., a DNA or RNA molecule from its natural cellular environment, and from association with other components of the cell, such as nucleic acid or polypeptide, so that it can be sequenced, replicated, and/or expressed. Thus, the RNA or DNA is “isolated” in that it is free from at least one contaminating nucleic acid with which it is normally associated in the natural source of the RNA or DNA and is preferably substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell, e.g., in a vector or plasmid.

As used herein, the term “recombinant nucleic acid”, e.g., “recombinant DNA sequence or segment” refers to a nucleic acid, e.g., to DNA, that has been derived or isolated from any appropriate cellular source, that may be subsequently chemically altered in vitro, so that its sequence is not naturally occurring, or corresponds to naturally occurring sequences that are not positioned as they would be positioned in a genome which has not been transformed with exogenous DNA. An example of preselected DNA “derived” from a source, would be a DNA sequence that is identified as a useful fragment within a given organism, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

Thus, recovery or isolation of a given fragment of DNA from a restriction digest can employ separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. Therefore, “recombinant DNA” includes completely synthetic DNA sequences, semi-synthetic DNA sequences, DNA sequences isolated from biological sources, and DNA sequences derived from RNA, as well as mixtures thereof.

Nucleic acid molecules having base substitutions (i.e., variants) are prepared by a variety of methods known in the art. These methods include, but are not limited to, isolation from a natural source (in the case of naturally occurring sequence variants) or preparation by oligonucleotide-mediated (or site-directed) mutagenesis, PCR mutagenesis, and cassette mutagenesis of an earlier prepared variant or a non-variant version of the nucleic acid molecule.

Oligonucleotide-mediated mutagenesis is a method for preparing substitution variants. Briefly, a nucleic acid encoding a protein described herein can be altered by hybridizing an oligonucleotide encoding the desired mutation to a DNA template, where the template is the single-stranded form of a plasmid or bacteriophage containing the unaltered or native gene sequence. After hybridization, a DNA polymerase is used to synthesize an entire second complementary strand of the template that will thus incorporate the oligonucleotide primer, and will code for the selected alteration in the nucleic acid encoding the protein. Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art.

The DNA template can be generated by those vectors that are either derived from bacteriophage M13 vectors (the commercially available M13mp18 and M13mp19 vectors are suitable), or those vectors that contain a single-stranded phage origin of replication. Thus, the DNA that is to be mutated may be inserted into one of these vectors to generate single-stranded template. Production of the single-stranded template is described in Chapter 3 of Sambrook and Russell, 2001. Alternatively, single-stranded DNA template may be generated by denaturing double-stranded plasmid (or other) DNA using standard techniques.

For alteration of the native DNA sequence (to generate amino acid sequence variants, for example), the oligonucleotide is hybridized to the single-stranded template under suitable hybridization conditions. A DNA polymerizing enzyme, usually the Klenow fragment of DNA polymerase I, is then added to synthesize the complementary strand of the template using the oligonucleotide as a primer for synthesis. A heteroduplex molecule is thus formed such that one strand of DNA encodes the mutated form of the DNA, and the other strand (the original template) encodes the native, unaltered sequence of the DNA. This heteroduplex molecule is then transformed into a suitable host cell, usually a prokaryote such as E. coli JM101. After the cells are grown, they are plated onto agarose plates and screened using the oligonucleotide primer radiolabeled with 32-phosphate to identify the bacterial colonies that contain the mutated DNA. The mutated region is then removed and placed in an appropriate vector, generally an expression vector of the type typically employed for transformation of an appropriate host.

The method described immediately above may be modified such that a homoduplex molecule is created wherein both strands of the plasmid contain the mutations(s). The modifications are as follows: The single-stranded oligonucleotide is annealed to the single-stranded template as described above. A mixture of three deoxyribonucleotides, deoxyriboadenosine (dATP), deoxyriboguanosine (dGTP), and deoxyribothymidine (dTTP), is combined with a modified thiodeoxyribocytosine called dCTP-(*S) (which can be obtained from the Amersham Corporation). This mixture is added to the template-oligonucleotide complex. Upon addition of DNA polymerase to this mixture, a strand of DNA identical to the template except for the mutated bases is generated. In addition, this new strand of DNA will contain dCTP-(*S) instead of dCTP, which serves to protect it from restriction endonuclease digestion.

After the template strand of the double-stranded heteroduplex is nicked with an appropriate restriction enzyme, the template strand can be digested with ExoIII nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell such as E. coli JM101.

### Expression Cassettes of the Invention

To prepare expression cassettes, the recombinant DNA sequence or segment may be circular or linear, double-stranded or single-stranded. Generally, the DNA sequence or segment is in the form of chimeric DNA, such as plasmid DNA or a vector that can also contain coding regions flanked by control sequences that promote the expression of the recombinant DNA present in the resultant transformed cell.

A “chimeric” vector or expression cassette, as used herein, means a vector or cassette including nucleic acid sequences from at least two different species, or has a nucleic acid sequence from the same species that is linked or associated in a manner that does not occur in the “native” or wild type of the species.

Aside from recombinant DNA sequences that serve as transcription units for an RNA transcript, or portions thereof, a portion of the recombinant DNA may be untranscribed, serving a regulatory or a structural function. For example, the recombinant DNA may have a promoter that is active in mammalian cells.

Other elements functional in the host cells, such as introns, enhancers, polyadenylation sequences and the like, may also be a part of the recombinant DNA. Such elements may or may not be necessary for the function of the DNA, but may provide improved expression of the DNA by affecting transcription, stability of the RNA, or the like. Such elements may be included in the DNA as desired to obtain the optimal expression in the cell.

Control sequences are DNA sequences necessary for the expression of an operably linked coding sequence in a particular host organism. The control sequences that are suitable for prokaryotic cells, for example, include a promoter, and optionally an operator sequence, and a ribosome binding site. Eukaryotic cells are known to utilize promoters, polyadenylation signals, and enhancers.

Operably linked nucleic acids are nucleic acids placed in a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Generally, operably linked DNA sequences are DNA sequences that are linked are contiguous. However, enhancers do not have to be contiguous. Linking is accomplished by ligation at convenient restriction sites. If such sites do not exist, the synthetic oligonucleotide adaptors or linkers are used in accord with conventional practice.

The recombinant DNA to be introduced into the cells may contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. For example, reporter genes include the chloramphenicol acetyl transferase gene (cat) from Tn9 of E. coli and the luciferase gene from firefly Photinus pyralis. Expression of the reporter gene is assayed at a suitable time after the DNA has been introduced into the recipient cells.

The general methods for constructing recombinant DNA that can transfect target cells are well known to those skilled in the art, and the same compositions and methods of construction may be utilized to produce the DNA useful herein. For example, Sambrook and Russell, infra, provides suitable methods of construction.

The recombinant DNA can be readily introduced into the host cells, e.g., mammalian, bacterial, yeast or insect cells by transfection with an expression vector composed of DNA encoding the protein (e.g., synthetase) by any procedure useful for the introduction into a particular cell, e.g., physical or biological methods, to yield a cell having the recombinant DNA stably integrated into its genome or existing as a episomal element, so that the DNA molecules, or sequences of the present invention are expressed by the host cell. Preferably, the DNA is introduced into host cells via a vector. The host cell is preferably of eukaryotic origin, e.g., plant, mammalian, insect, yeast or fungal sources, but host cells of non-eukaryotic origin may also be employed.

Physical methods to introduce a preselected DNA into a host cell include calcium phosphate precipitation, lipofection, particle bombardment, microinjection, electroporation, and the like. Biological methods to introduce the DNA of interest into a host cell include the use of DNA and RNA viral vectors. For mammalian gene therapy, as described herein below, it is desirable to use an efficient means of inserting a copy gene into the host genome. Viral vectors, and especially retroviral vectors, have become the most widely used method for inserting genes into mammalian, e.g., human cells. Other viral vectors can be derived from poxviruses, herpes simplex virus I, adenoviruses and adeno-associated viruses, and the like.

As discussed above, a “transfected”, “or “transduced” host cell or cell line is one in which the genome has been altered or augmented by the presence of at least one heterologous or recombinant nucleic acid sequence. The host cells of the present invention are typically produced by transfection with a DNA sequence in a plasmid expression vector, a viral expression vector, or as an isolated linear DNA sequence. The transfected DNA can become a chromosomally integrated recombinant DNA sequence, which is composed of sequence encoding the protein (e.g., a synthetase).

To confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular protein, e.g., by immunological means (ELISAs and Western blots) or by assays described herein to identify agents falling within the scope of the invention.

To detect and quantitate RNA produced from introduced recombinant DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the recombinant DNA segment in question, they do not provide information as to whether the preselected DNA segment is being expressed. Expression may be evaluated by specifically identifying the protein products of the introduced recombinant DNA sequences or evaluating the phenotypic changes brought about by the expression of the introduced recombinant DNA segment in the host cell.

The instant invention provides a cell expression system for expressing exogenous nucleic acid material in a mammalian recipient. The expression system, also referred to as a “genetically modified cell”, comprises a cell and an expression vector for expressing the exogenous nucleic acid material. The genetically modified cells are suitable for administration to a mammalian recipient, where they replace the endogenous cells of the recipient. Thus, the preferred genetically modified cells are non-immortalized and are non-tumorigenic.

According to one embodiment, the cells are transfected or otherwise genetically modified ex vivo. The cells are isolated from a mammal (preferably a human), nucleic acid introduced (i.e., transduced or transfected in vitro) with a vector for expressing a heterologous (e.g., recombinant) gene encoding the therapeutic agent, and then administered to a mammalian recipient for delivery of the therapeutic agent in situ. The mammalian recipient may be a human and the cells to be modified are autologous cells, i.e., the cells are isolated from the mammalian recipient.

According to another embodiment, the cells are transfected or transduced or otherwise genetically modified in vivo. The cells from the mammalian recipient are transduced or transfected in vivo with a vector containing exogenous nucleic acid material for expressing a heterologous (e.g., recombinant) gene encoding a therapeutic agent and the therapeutic agent is delivered in situ.

As used herein, “exogenous nucleic acid material” refers to a nucleic acid or an oligonucleotide, either natural or synthetic, which is not naturally found in the cells; or if it is naturally found in the cells, is modified from its original or native form. Thus, “exogenous nucleic acid material” includes, for example, a non-naturally occurring nucleic acid that can be transcribed into a “heterologous gene” (i.e., a gene encoding a protein that is not expressed or is expressed at biologically insignificant levels in a naturally-occurring cell of the same type). To illustrate, a synthetic or natural gene encoding human erythropoietin (EPO) would be considered “exogenous nucleic acid material” with respect to human peritoneal mesothelial cells since the latter cells do not naturally express EPO. Still another example of “exogenous nucleic acid material” is the introduction of only part of a gene to create a recombinant gene, such as combining a regulatable promoter with an endogenous coding sequence via homologous recombination.

### Methods for Introducing the Expression Cassettes of the Invention into Cells

The condition amenable to gene therapy may be a prophylactic process, i.e., a process for preventing disease or an undesired medical condition. Thus, the instant invention embraces a system for delivering protein s, such as a synthetase, that have a prophylactic function (i.e., a prophylactic agent) to the mammalian recipient.

The nucleic acid material (e.g., an expression cassette encoding a synthetase) can be introduced into the cell ex vivo or in vivo by genetic transfer methods, such as transfection or transduction, to provide a genetically modified cell. Various expression vectors (i.e., vehicles for facilitating delivery of exogenous nucleic acid into a target cell) are known to one of ordinary skill in the art.

As used herein, “transfection of cells” refers to the acquisition by a cell of new nucleic acid material by incorporation of added DNA. Thus, transfection refers to the insertion of nucleic acid into a cell using physical or chemical methods. Several transfection techniques are known to those of ordinary skill in the art including calcium phosphate DNA co-precipitation, DEAE-dextran, electroporation, cationic liposome-mediated transfection, tungsten particle-facilitated microparticle bombardment, and strontium phosphate DNA co-precipitation.

In contrast, “transduction of cells” refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous nucleic acid material contained within the retrovirus is incorporated into the genome of the transduced cell. A cell that has been transduced with a chimeric DNA virus (e.g., an adenovirus carrying a cDNA encoding a therapeutic agent), will not have the exogenous nucleic acid material incorporated into its genome but will be capable of expressing the exogenous nucleic acid material that is retained extrachromosomally within the cell.

The exogenous nucleic acid material can include the nucleic acid encoding the protein together with a promoter to control transcription. The promoter characteristically has a specific nucleotide sequence necessary to initiate transcription. The exogenous nucleic acid material may further include additional sequences (i.e., enhancers) required to obtain the desired gene transcription activity. For the purpose of this discussion an “enhancer” is simply any non-translated DNA sequence that works with the coding sequence (in cis) to change the basal transcription level dictated by the promoter. The exogenous nucleic acid material may be introduced into the cell genome immediately downstream from the promoter so that the promoter and coding sequence are operatively linked so as to permit transcription of the coding sequence. An expression vector can include an exogenous promoter element to control transcription of the inserted exogenous gene. Such exogenous promoters include both constitutive and regulatable promoters.

Naturally-occurring constitutive promoters control the expression of essential cell functions. As a result, a nucleic acid sequence under the control of a constitutive promoter is expressed under all conditions of cell growth. Constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the beta-actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others.

Nucleic acid sequences that are under the control of regulatable promoters are expressed only or to a greater or lesser degree in the presence of an inducing or repressing agent, (e.g., transcription under control of the metallothionein promoter is greatly increased in presence of certain metal ions). Regulatable promoters include responsive elements (REs) that stimulate transcription when their inducing factors are bound. For example, there are REs for serum factors, steroid hormones, retinoic acid, cyclic AMP, and tetracycline and doxycycline. Promoters containing a particular RE can be chosen in order to obtain a regulatable response and in some cases, the RE itself may be attached to a different promoter, thereby conferring regulatability to the encoded nucleic acid sequence. Thus, by selecting the appropriate promoter (constitutive versus regulatable; strong versus weak), it is possible to control both the existence and level of expression of a nucleic acid sequence in the genetically modified cell. If the nucleic acid sequence is under the control of an regulatable promoter, delivery of the therapeutic agent in situ is triggered by exposing the genetically modified cell in situ to conditions for permitting transcription of the nucleic acid sequence, e.g., by intraperitoneal injection of specific inducers of the regulatable promoters which control transcription of the agent. For example, in situ expression of a nucleic acid sequence under the control of the metallothionein promoter in genetically modified cells is enhanced by contacting the genetically modified cells with a solution containing the appropriate (i.e., inducing) metal ions in situ.

Accordingly, the amount of protein (e.g., a synthetase) generated in situ is regulated by controlling such factors as the nature of the promoter used to direct transcription of the nucleic acid sequence, (i.e., whether the promoter is constitutive or regulatable, strong or weak) and the number of copies of the exogenous nucleic acid sequence encoding the protein that are in the cell.

In one embodiment of the present invention, an expression cassette may contain a pol II promoter that is operably linked to a nucleic acid sequence encoding a protein (e.g., a synthetase). Thus, the pol II promoter, i.e., a RNA polymerase II dependent promoter, initiates the transcription of the RNA, which encodes the protein of interest. In another embodiment, the pol II promoter is regulatable.

A pol II promoter may be used in its entirety, or a portion or fragment of the promoter sequence may be used in which the portion maintains the promoter activity. As discussed herein, pol II promoters are known to a skilled person in the art and include the promoter of any protein-encoding gene, e.g., an endogenously regulated gene or a constitutively expressed gene. For example, the promoters of genes regulated by cellular physiological events, e.g., heat shock, oxygen levels and/or carbon monoxide levels, e.g., in hypoxia, may be used in the expression cassettes of the invention. In addition, the promoter of any gene regulated by the presence of a pharmacological agent, e.g., tetracycline and derivatives thereof, as well as heavy metal ions and hormones may be employed in the expression cassettes of the invention. In an embodiment of the invention, the pol II promoter can be the CMV promoter or the RSV promoter. In another embodiment, the pol II promoter is the CMV promoter.

As discussed above, a pol II promoter of the invention may be one naturally associated with an endogenously regulated gene or sequence, as may be obtained by isolating the 5′ non-coding sequences located upstream of the coding segment and/or exon. The pol II promoter of the expression cassette can be, for example, the same pol II promoter driving expression of the targeted gene of interest. Alternatively, the nucleic acid sequence encoding the protein may be placed under the control of a recombinant or heterologous pol II promoter, which refers to a promoter that is not normally associated with the targeted gene's natural environment. Such promoters include promoters isolated from any eukaryotic cell, and promoters not “naturally occurring,” i.e., containing different elements of different transcriptional regulatory regions, and/or mutations that alter expression. In addition to producing nucleic acid sequences of promoters synthetically, sequences may be produced using recombinant cloning and/or nucleic acid amplification technology, including PCR, in connection with the compositions disclosed herein.

In one embodiment, a pol II promoter that effectively directs the expression of the RNA in the cell type, organelle, and organism chosen for expression will be employed. Those of ordinary skill in the art of molecular biology generally know the use of promoters for protein expression, for example, see Sambrook and Russell (2001), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The identity of tissue-specific promoters, as well as assays to characterize their activity, is well known to those of ordinary skill in the art.

In addition to at least one promoter and at least one heterologous nucleic acid sequence encoding the protein, the expression vector may include a selection gene, for example, a neomycin resistance gene, for facilitating selection of cells that have been transfected or transduced with the expression vector.

Cells can also be transfected with two or more expression vectors, at least one vector containing the nucleic acid sequence(s) encoding the protein(s), the other vector containing a selection gene. The selection of a suitable promoter, enhancer, selection gene and/or signal sequence is deemed to be within the scope of one of ordinary skill in the art without undue experimentation.

The following discussion is directed to various utilities of the instant invention. For example, the instant invention has utility as an expression system suitable for expressing a protein described herein.

The instant invention also provides methods for genetically modifying cells of a mammalian recipient in vivo. According to one embodiment, the method comprises introducing an expression vector for expressing a protein described herein in cells of the mammalian recipient in situ by, for example, injecting the vector into the recipient.

### Delivery Vehicles for the Expression Cassettes of the Invention

Delivery of compounds into tissues and across the blood-brain barrier can be limited by the size and biochemical properties of the compounds. Currently, efficient delivery of compounds into cells in vivo can be achieved only when the molecules are small (usually less than 600 Daltons). Gene transfer for the treatment of cancer has been accomplished with recombinant adenoviral vectors.

The selection and optimization of a particular expression vector for expressing a specific protein (e.g., a synthetase) in a cell can be accomplished by obtaining the nucleic acid sequence encoding the protein, possibly with one or more appropriate control regions (e.g., promoter, insertion sequence); preparing a vector construct comprising the vector into which is inserted the nucleic acid sequence encoding the protein; transfecting or transducing cultured cells in vitro with the vector construct; and determining whether the protein is present in the cultured cells.

Vectors for cell gene therapy include viruses, such as replication-deficient viruses (described in detail below). Exemplary viral vectors are derived from Harvey Sarcoma virus, ROUS Sarcoma virus, (MPSV), Moloney murine leukemia virus and DNA viruses (e.g., adenovirus).

Replication-deficient retroviruses are capable of directing synthesis of all virion proteins, but are incapable of making infectious particles. Accordingly, these genetically altered retroviral expression vectors have general utility for high-efficiency transduction of nucleic acid sequences in cultured cells, and specific utility for use in the method of the present invention. Such retroviruses further have utility for the efficient transduction of nucleic acid sequences into cells in vivo. Retroviruses have been used extensively for transferring nucleic acid material into cells. Protocols for producing replication-deficient retroviruses (including the steps of incorporation of exogenous nucleic acid material into a plasmid, transfection of a packaging cell line with plasmid, production of recombinant retroviruses by the packaging cell line, collection of viral particles from tissue culture media, and infection of the target cells with the viral particles) are well known in the art.

An advantage of using retroviruses for gene therapy is that the viruses insert the nucleic acid sequence encoding the protein into the host cell genome, thereby permitting the nucleic acid sequence encoding the protein to be passed on to the progeny of the cell when it divides. Promoter sequences in the LTR region have can enhance expression of an inserted coding sequence in a variety of cell types. Some disadvantages of using a retrovirus expression vector are (1) insertional mutagenesis, i.e., the insertion of the nucleic acid sequence encoding the protein into an undesirable position in the target cell genome which, for example, leads to unregulated cell growth and (2) the need for target cell proliferation in order for the nucleic acid sequence encoding the protein carried by the vector to be integrated into the target genome.

Another viral candidate useful as an expression vector for transformation of cells is the adenovirus, a double-stranded DNA virus. The adenovirus is infective in a wide range of cell types, including, for example, muscle and endothelial cells.

Adenoviruses (Ad) are double-stranded linear DNA viruses with a 36 kb genome. Several features of adenovirus have made them useful as transgene delivery vehicles for therapeutic applications, such as facilitating in vivo gene delivery. Recombinant adenovirus vectors have been shown to be capable of efficient in situ gene transfer to parenchymal cells of various organs, including the lung, brain, pancreas, gallbladder, and liver. This has allowed the use of these vectors in methods for treating inherited genetic diseases, such as cystic fibrosis, where vectors may be delivered to a target organ. In addition, the ability of the adenovirus vector to accomplish in situ tumor transduction has allowed the development of a variety of anticancer gene therapy methods for non-disseminated disease. In these methods, vector containment favors tumor cell-specific transduction.

Like the retrovirus, the adenovirus genome is adaptable for use as an expression vector for gene therapy, i.e., by removing the genetic information that controls production of the virus itself. Because the adenovirus functions in an extrachromosomal fashion, the recombinant adenovirus does not have the theoretical problem of insertional mutagenesis.

Several approaches traditionally have been used to generate the recombinant adenoviruses. One approach involves direct ligation of restriction endonuclease fragments containing a nucleic acid sequence of interest to portions of the adenoviral genome. Alternatively, the nucleic acid sequence of interest may be inserted into a defective adenovirus by homologous recombination results. The desired recombinants are identified by screening individual plaques generated in a lawn of complementation cells.

Most adenovirus vectors are based on the adenovirus type 5 (Ad5) backbone in which an expression cassette containing the nucleic acid sequence of interest has been introduced in place of the early region 1 (E1) or early region 3 (E3). Viruses in which E1 has been deleted are defective for replication and are propagated in human complementation cells (e.g., 293 or 911 cells), which supply the missing gene E1 and pIX in trans.

In one embodiment of the present invention, one will desire to generate the protein (e.g., a synthetase) in a CNS cancer tumor (e.g, a glioma). A suitable vector for this application is an FIV vector or an AAV vector. For example, one may use AAV5. Also, one may apply poliovirus or HSV vectors.

Recombinant adenovirus, adeno-associated virus (AAV) and feline immunodeficiency virus (FIV) can be used to deliver genes in vitro and in vivo. Each has its own advantages and disadvantages. Adenoviruses are double stranded DNA viruses with large genomes (36 kb) and have been engineered to accommodate expression cassettes in distinct regions.

Adeno-associated viruses have encapsidated genomes, similar to Ad, but are smaller in size and packaging capacity (˜30 nm vs. ˜100 nm; packaging limit of ˜4.5 kb). AAV contain single stranded DNA genomes of the + or the − strand. Eight serotypes of AAV (1-8) have been studied extensively, three of which have been evaluated in the brain. An important consideration for the present application is that AAV5 transduces striatal and cortical neurons, and is not associated with any known pathologies.

FIV is an enveloped virus with a strong safety profile in humans; individuals bitten or scratched by FIV-infected cats do not seroconvert and have not been reported to show any signs of disease. Like AAV, FIV provides lasting transgene expression in mouse and nonhuman primate neurons, and transduction can be directed to different cell types by pseudotyping, the process of exchanging the viruses' native envelope for an envelope from another virus.

Thus, as will be apparent to one of ordinary skill in the art, a variety of suitable viral expression vectors are available for transferring exogenous nucleic acid material into cells. The selection of an appropriate expression vector to express a therapeutic agent for a particular condition amenable to gene therapy and the optimization of the conditions for insertion of the selected expression vector into the cell, are within the scope of one of ordinary skill in the art without the need for undue experimentation.

In another embodiment, the expression vector is in the form of a plasmid, which is transferred into the target cells by one of a variety of methods: physical (e.g., microinjection, electroporation, scrape loading, microparticle bombardment) or by cellular uptake as a chemical complex (e.g., calcium or strontium co-precipitation, complexation with lipid, complexation with ligand). Several commercial products are available for cationic liposome complexation including Lipofectin™ (Gibco-BRL, Gaithersburg, Md.) and Transfectam™ (ProMega, Madison, Wis.). However, the efficiency of transfection by these methods is highly dependent on the nature of the target cell and accordingly, the conditions for optimal transfection of nucleic acids into cells using the above-mentioned procedures must be optimized. Such optimization is within the scope of one of ordinary skill in the art without the need for undue experimentation.

Certain embodiments of the invention will now be illustrated by the following non-limiting Examples.

### EXAMPLE 1

### Site-Specific Encoding of Fluorinated Phenylalanine Residues in Bacterial and Mammalian Cells

The aromatic side-chains of phenylalanine, tyrosine, and tryptophan interact with their environments via both hydrophobic and electrostatic interactions. Determining the extent to which these contribute to protein function and stability is not possible with conventional mutagenesis. Serial fluorination of a given aromatic is a validated method in vitro and in silico to specifically alter electrostatic characteristics, but this approach is restricted to a select few experimental systems. Here, we report a new group of pyrrolysine-based aminoacyl-tRNA synthetase/tRNA pairs that enable the site-specific encoding of a varied spectrum of fluorinated phenylalanine amino acids in E. coli and mammalian (HEK 293T) cells. By allowing the cross-kingdom expression of proteins bearing these unnatural amino acids at biochemical scale, these tools will enable deconstruction of biological mechanisms which utilize aromatic-pi interactions in structural and cellular contexts.

The aromatic side-chains of phenylalanine, tyrosine, and tryptophan are crucial for protein function and pharmacology due to their hydrophobic and electrostatic contributions to catalytic centers and ligand-binding pockets. However, few experimental approaches can chemically assess the functional roles of aromatics in cellular environments. The accepted computational method for aromatic interrogation is via serial fluorination, which lacks an experimental correlate in bacterial or mammalian cell systems. We have identified a family of synthetases to encode multiple different types of fluorinated phenylalanine residues in E. coli and HEK cells via nonsense suppression. The efficiency of these synthetases is sufficient to support biochemical characterization and structural determination of proteins with site-specific incorporation of unnatural phenylalanine analogs.

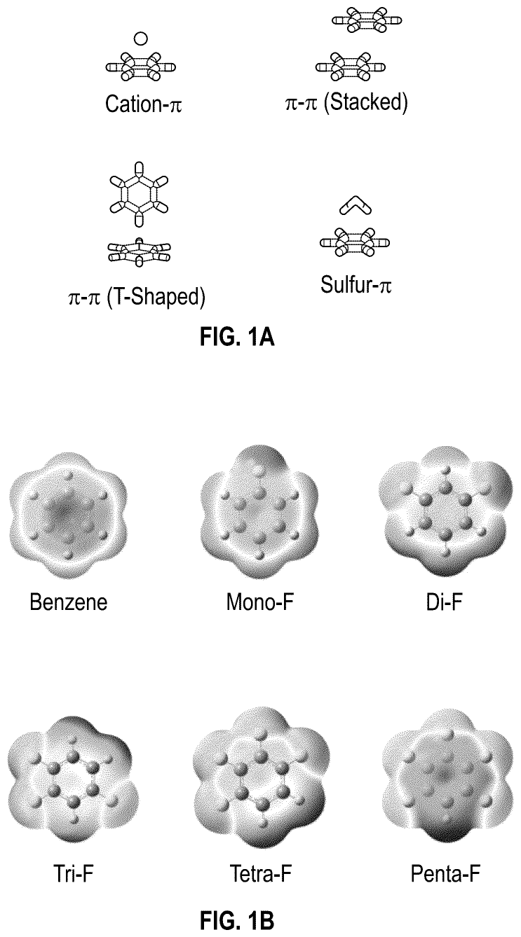

While primarily appreciated for its hydrophobicity, the benzyl side chain of phenylalanine also engages in various types of energetically favorable aromatic interactions. The phenylalanine side chain engages in various aromatic/electrostatic interactions that play roles in protein structure and ligand interactions (FIG. 1A). Energetically significant aromatic interactions involving phenylalanine are believed to be widespread in nature. Based on emerging structural data, they have been increasingly proposed to play important mechanistic roles in ligand recognition, and protein-protein interactions, both in normal function and as a result of clinical mutation. They may also play important roles in membrane anchoring, via attraction of the benzyl side chain to choline lipid headgroups. Testing the specific functional role for aromaticity of the benzyl side chain in an interaction is not possible using conventional mutagenesis since mutation to a non-aromatic natural amino acid also causes concomitant changes in amino acid shape, convoluting data interpretation. Fluorination of the aromatic ring, on the other hand, allows for redistribution of the electrostatic potential of Phe with only minor steric changes (FIG. 1B), thus allowing the specific testing of the functional relevance of aromaticity. Increasing the number of fluorine substitutions results in a linear decrease in the electrostatic potential for pi interactions.

The site-specific installation of fluorinated phenylalanine amino acids can be accomplished via protein synthesis and by the direct injection of misacylated nonsense suppressor tRNA, the latter of which has been used to functionally validate and quantify aromatic interactions in some membrane proteins, including ion channels. These methods, while chemically flexible, cannot be easily scaled for biochemical and structural approaches. Additionally, some natural amino acids have been replaced with fluorinated, non-canonical structural analogs via permissive endogenous synthetases, but these methods are not site-specific, resulting in multiple sites within the protein sequence being modified heterogeneously. On the other hand, nonsense suppression via co-evolved aminoacyl tRNA-synthetase/tRNA pairs allows for biochemical-scale production of proteins bearing site-specifically installed non-canonical amino acids (ncAA) in diverse cell systems and even organisms. Previously, one such tRNA-synthetase/tRNA pair that encodes select fluorinated phenylalanine analogs into proteins was selected by rational design followed by screening of a single-residue library of 20 variants. This system was shown functional in E. coli if grown in minimal media without exogenous phenylalanine but the lack of a sufficiently efficient tRNA-synthetase/tRNA pair stymies field progress. A method to accurately encode fluorinated Phe ncAAs in complex media and in eukaryotic cells would prove highly valuable for many clinically relevant protein targets, including the myriad genes that must be expressed and studied in mammalian cells.

We hypothesized that by screening a greater number of tRNA-synthetase active site variants (˜10⁷), using a novel strategy that enabled enrichment of variants able to selectively incorporate fluorinated phenylalanine derivatives but not phenylalanine, we may identify synthetases with the desired utility and versatility. We describe here the identification and validation of a group of synthetases, which we have named Pheₓ, from the pyrrolysine system which are competent for site-specific encoding of fluorinated di-, tri-, tetra, and penta-fluoro phenylalanine analogs within proteins in both bacterial and mammalian expression systems. Intact protein mass spectrometry defines the sythetase and ncAA combinations that enable high fidelity encoding at the amber suppression site without spurious substitution at natural codons. Finally, we demonstrate biochemical scale encoding in two large (>150 KDa) membrane proteins of high clinical interest.

### Results

### A Pyl-Based Screen to Identify Synthetase Variants that Encode Fluorinated Phenylalanine Analogs in E. coli

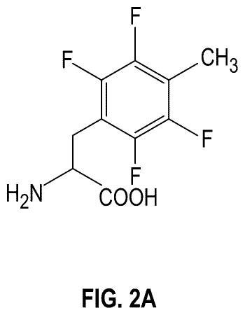

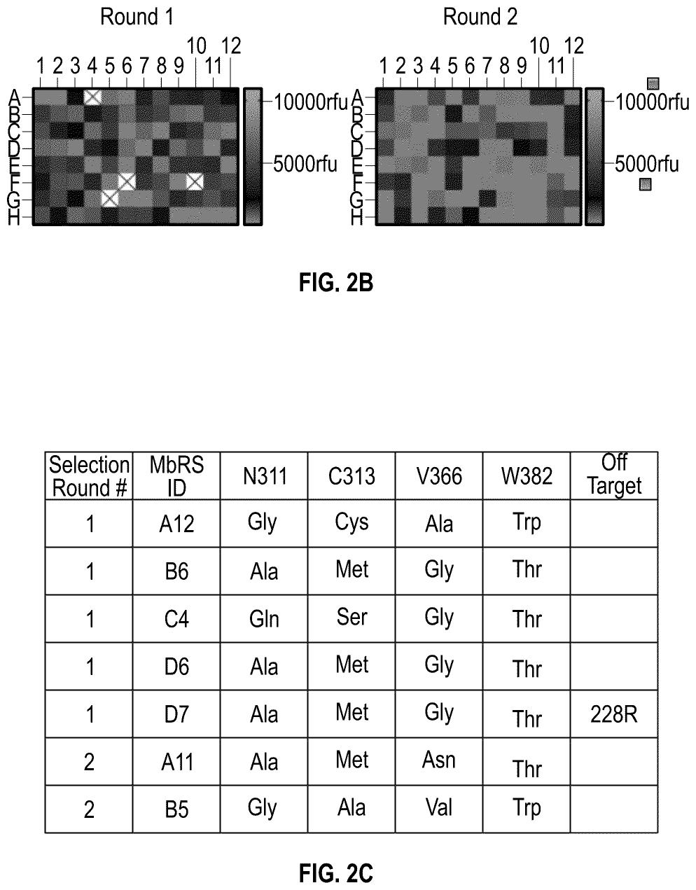

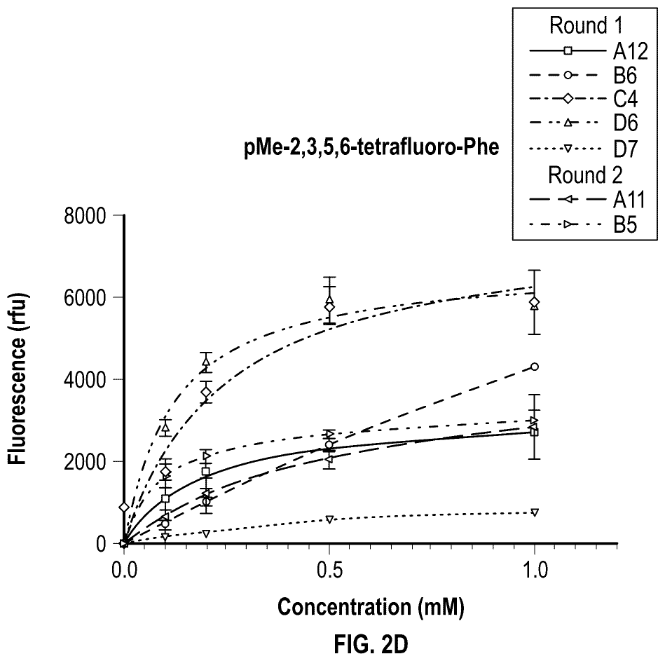

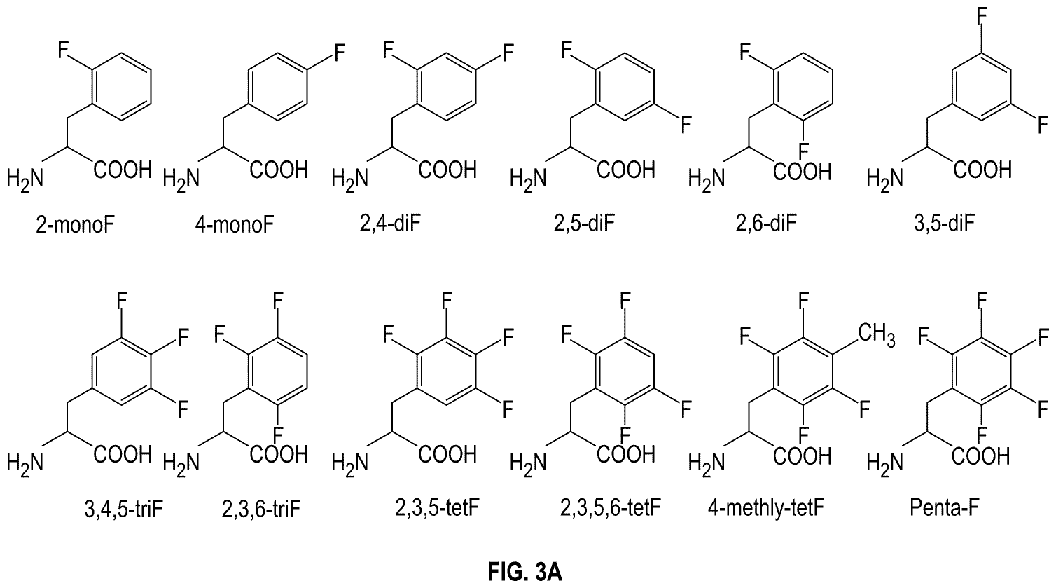

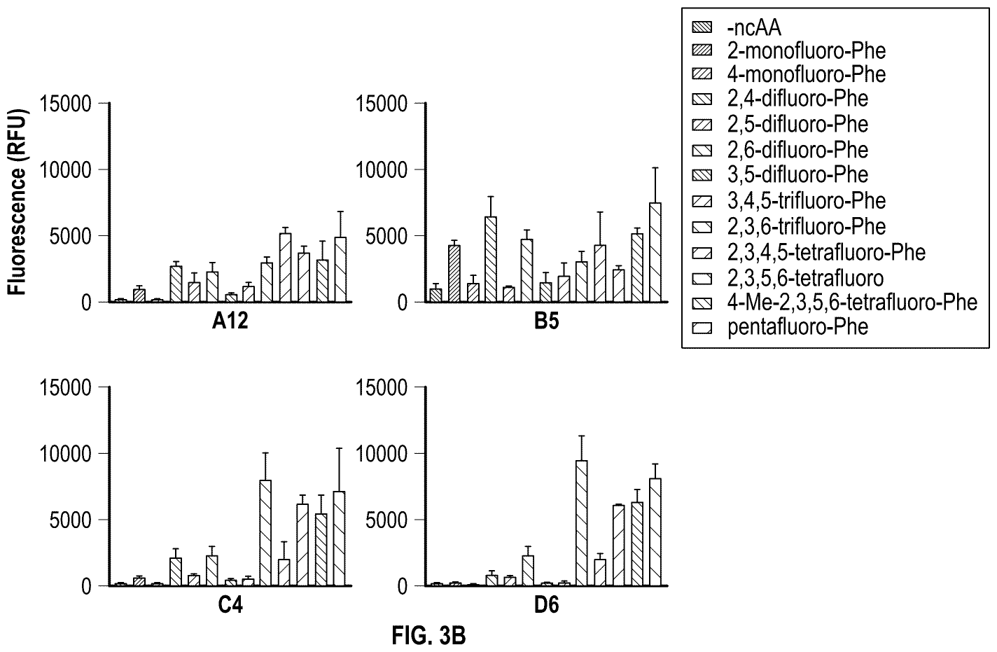

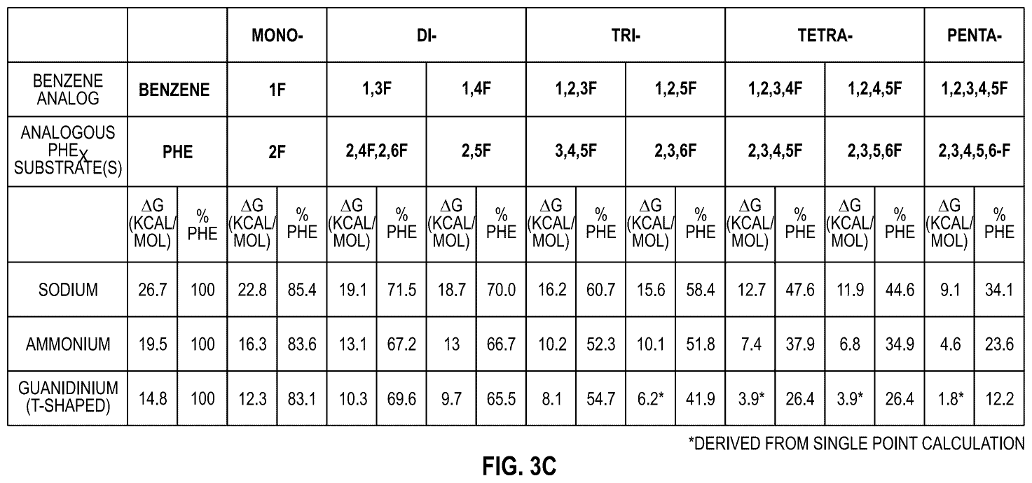

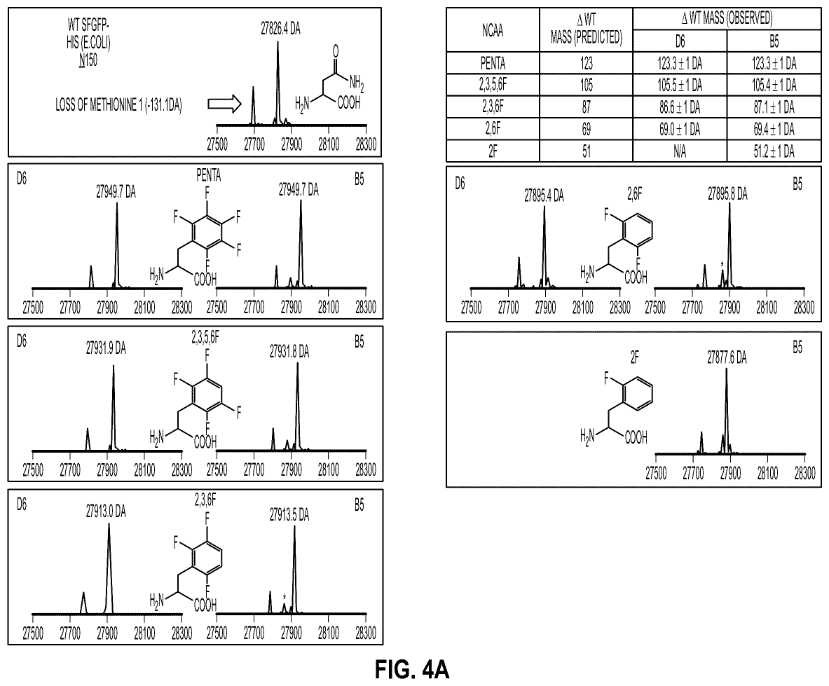

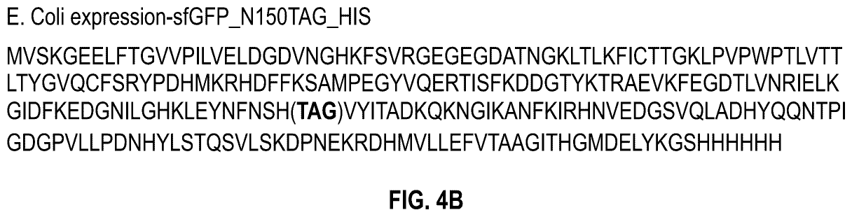

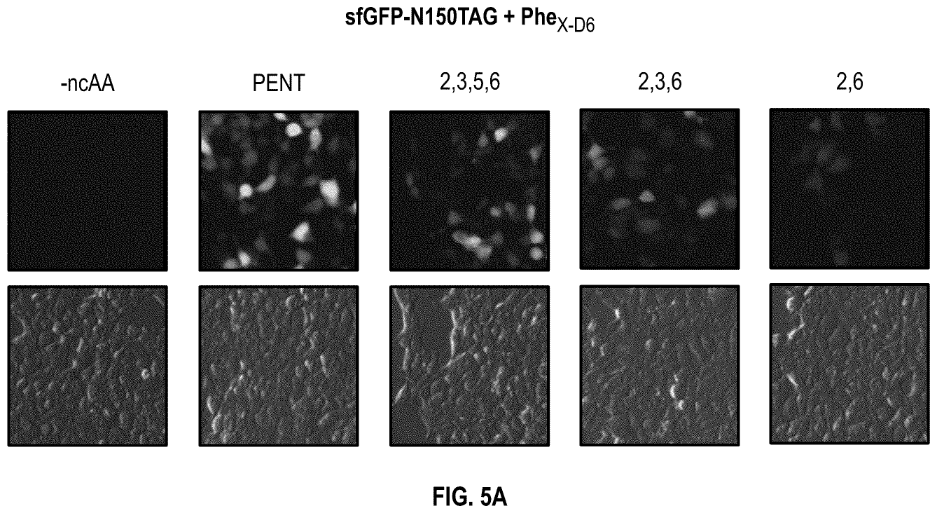

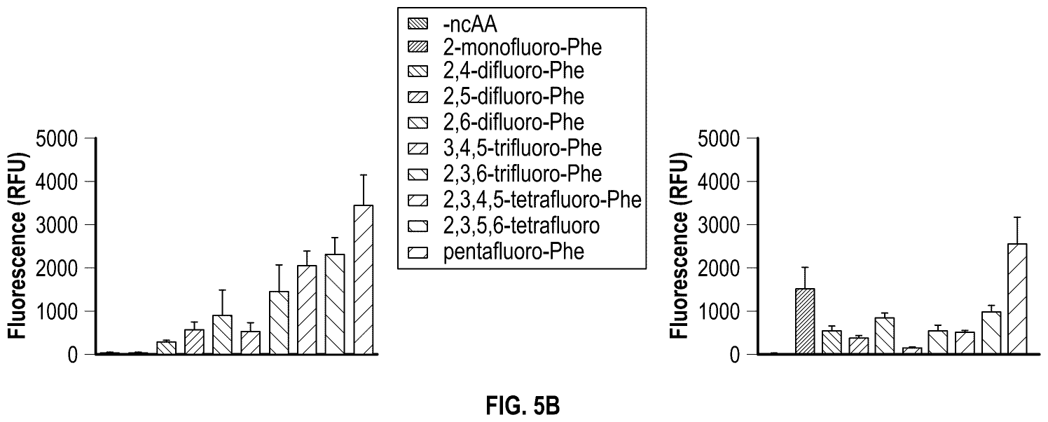

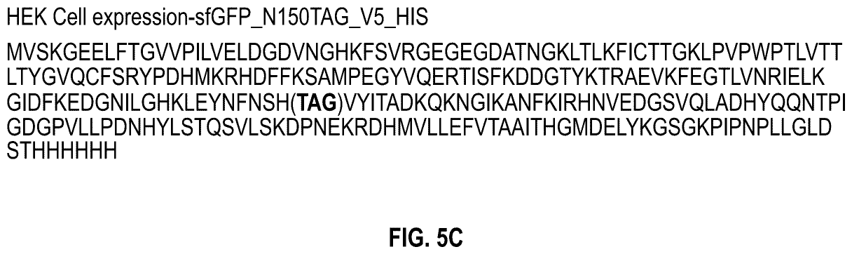

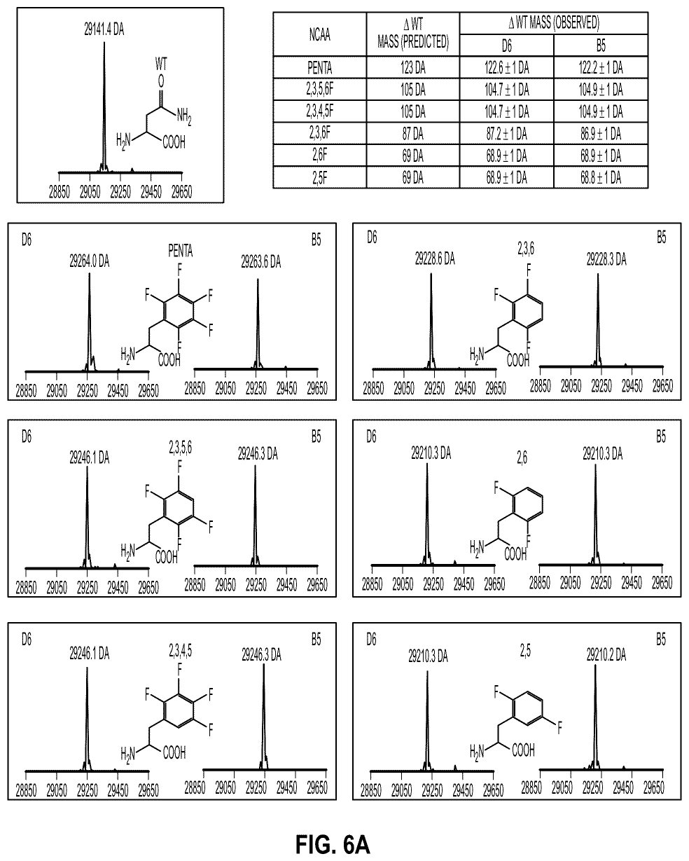

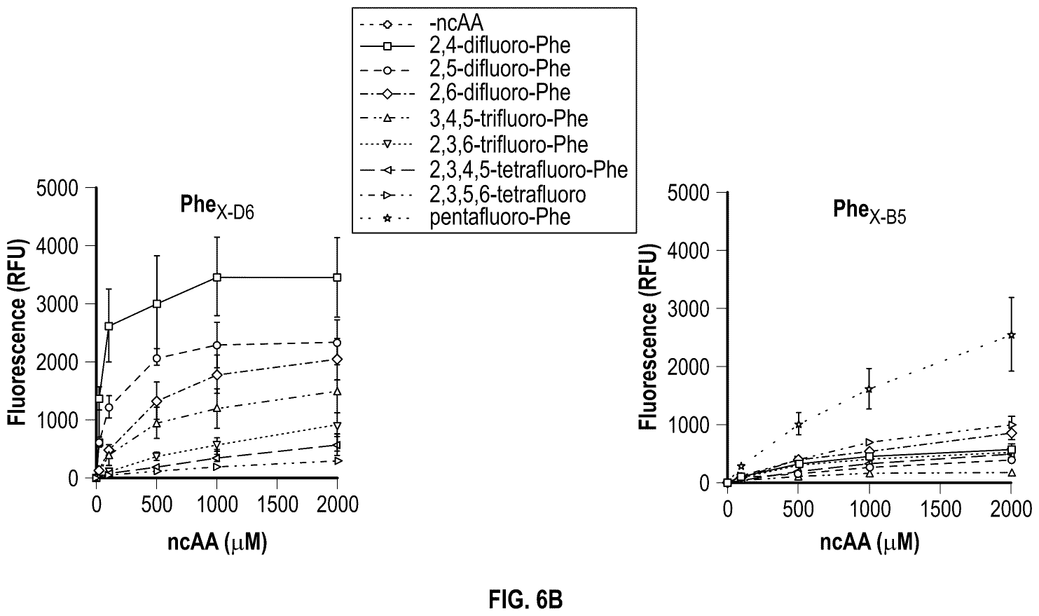

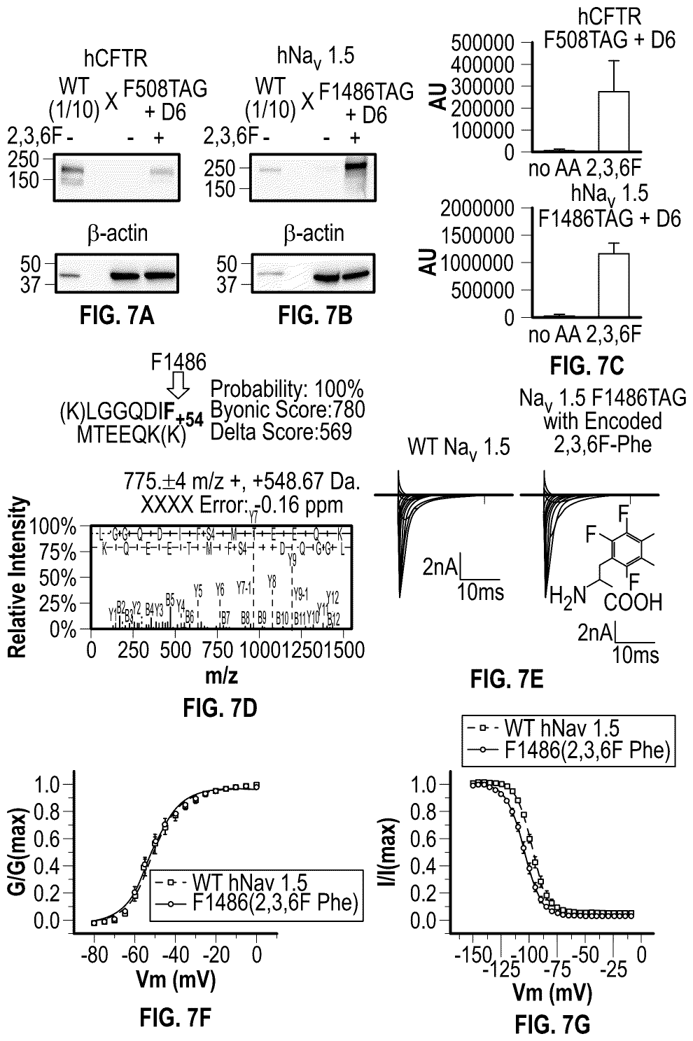

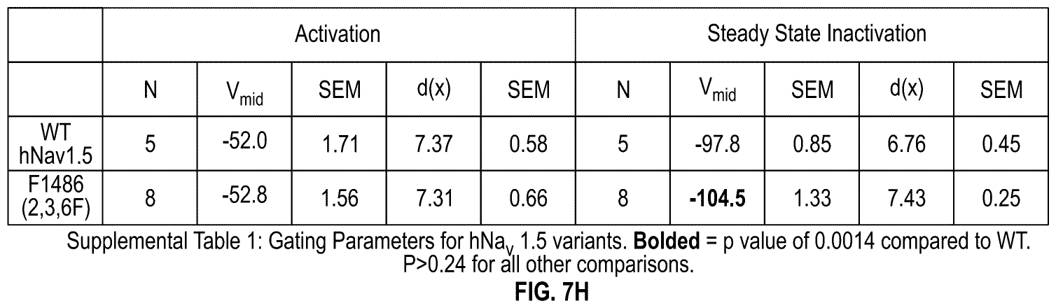

Historical attempts to screen for robust Pyl synthetases able to incorporate fluorinated phenylalanine analogs were not successful, possibly owing to the close steric resemblance of fluoro-benzene variants. In the present work, we decided to screen a library of Pyl synthetases with para-methyl, tetra-fluoro-phenylalanine (FIG. 2A), in the hope that the additional structural bulk of the methyl at the para position would permit identification of synthetases selective against natural amino acids but selective for fluorinated Phe derivatives, perhaps including those lacking the para-methyl group. Two independent rounds (FIG. 2B) of positive and negative screening within a Pyrrolysine-based aminoacyl tRNA synthetase library (see methods) in E. coli resulted in the identification of several synthetases that enabled amber suppression within superfolder GFP (sfGFP_N150TAG) in response to para-methyl tetra-fluoro Phe (FIG. 2C). Unique synthetases wherein suppression of sfGFP (from GFP signal) was greater than 10-fold above background are shown in FIG. 2C. As a preliminary assessment of the quality of the evolved tRNA/RS pair, experiments to determine the concentration of ncAA required to express ncAA-sfGFP at half its maximum expression level, called the Unnatural Protein 50% (UP50), were performed for the synthetases using para-methyl tetra-fluoro Phe (FIG. 2D). The UP50 values for para-methyl tetra-fluoro Phe for the selected tRNA/RS pairs validate they will function efficiently at 0.5 mM ncAA.

### Synthetases Selected for Para-Methyl-Tetra-Fluoro-Phe are Permissive to a Panel of Fluorinated Phe Analogs

An important purpose of the screening efforts was to identify synthetases that enable the encoding of fluorinated (but non-methylated) phenylalanine analogs within proteins. Therefore, we tested for permissivity of the selected synthetases against a diverse collection of phenylalanine analogs (FIG. 3A). In practice, this was an iterative process, with preliminary results from both E. coli and HEK cells motivating the synthesis and evaluation of additional structural analogs to audition in both systems. Interestingly, the synthetases tested encoded penta-fluoro Phe and 2,3,5,6 tetra-fluoro Phe at comparable levels to para-methyl tetra-fluoro Phe in E. coli, indicating that the para-methyl group is not required for recognition (FIG. 3B). Each synthetase showed some degree of permissivity; Phe_{X-B5} and Phe_{X-D6} were particularly interesting examples with substantially different active site sequences (FIG. 2C) and recognition profiles (FIG. 3B). Phe_{X-B5} displayed broader utility, but also lower apparent specificity over background, than Phe_{X-D6} in E. coli.

With permissivity profiles in hand, we performed ab initio quantum calculations to determine the effect of specific fluorination patterns on the potential of each analog to engage in electrostatic interactions, which has been previously done for a few of these analogs. We calculated the theoretical ΔG (kcal/mol) for interactions with three different cations and their representative benzene structures (FIG. 3C). As expected, increasing the number of fluorine atoms elicited a linear decrease in the binding energy. FIG. 3C expresses both the derived ΔG values and how they compare as a percentage of the native benzene of Phe (% Phe). Importantly, the relative positioning of the fluorine atoms did not greatly affect energetics of binding, suggesting that the synthetases encode analogs with different potential local electrostatic effects but equivalent global perturbations. The encoding of multiple di-, tri- or tetra-fluoro Phe analogs may prove useful to better understand phenylalanine side chains involved in complex, or multivalent motifs.

We next verified the fidelity of the top tRNA/RS pairs by analyzing the intact sfGFP proteins by ESI-mass spectrometry to confirm accurate encoding of the fluorinated phenylalanine derivatives. We used the Phe_{X-D6} and Phe_{X-B5} synthetases to express and purify sfGFP_N150TAG_HIS in E. coli (FIG. 4B). Whole protein sfGFP masses were consistent with replacement of N150 with each of the respective F-Phe ncAAs. Using Phe_{X-D6}, we observed evidence of high-fidelity incorporation (FIG. 4A) for di-, tri-, tetra- and pentafluoro phenylalanine (see methods for additional details). When the Phe_{X-B5} synthetase was used to express sfGFP bearing a range of ncAAs at N150, evidence of efficient encoding was clear for a wide range of fluorinated ncAAs (FIG. 4A). However, for several ncAAs with lower encoding efficiency, detectable peaks corresponding to the mass of Phe at N150 were observed (FIG. 4A, Δ mass of +33 Da from asparagine, asterisks). This background encoding of natural AAs was expected from the higher fluorescence values in the absence of ncAA for Phe_{X-B5} suggesting that Phe_{X-B5} incorporates natural amino acids in E. coli when using ncAAs poorly recognized by the synthetase.

### Site-Specific Encoding of Fluoro-Phe ncAAs into Proteins in HEKT Cells

To evaluate the encoding of fluorinated analogs in HEK cells, we cloned the Phe_{X-B5} and Phe_{X-D6} synthetases into the pAcBac1 plasmid and co-transfected them with a plasmid encoding sfGFP_N150TAG in the presence or absence of fluoro-Phe ncAAs. In HEK cells both the Phe_{X-D6} and Phe_{X-B5} tRNA/RS pairs produced sfGFP in the presence of fluoro-Phe ncAAs but not appreciably in their absence, yielding green cells which were imaged for fluorescence at 24 hours (FIG. 5A). For quantitative comparison of ncAA-protein expression, fluorescence from cell lysate was measured. As with E. coli fluoro-Phe encoding, the position and number of fluorine substitutions affected the efficiency of producing ncAA-protein with the Phe_{X-D6} and Phe_{X-B5} tRNA/RS pairs (FIG. 5B). Of note, the relative fidelity of these tRNA/RS pairs was better in HEK cells compared to that in E. coli., but the background fluorescence from the Phe_{X-B5} tRNA/RS pair was still higher than Phe_{X-D6} (0.84% and 0.15% of maximum encoding for Penta-F Phe for Phe_{X-B5} and Phe_{X-D6} pairs respectively).

To rigorously confirm the efficiency and fidelity of these tRNA/RS pairs in a eukaryotic context, ncAA-sfGFP was expressed in HEKT cells at 2 mM ncAA, purified via c-terminal His₆ tag and subjected to ESI-mass spectroscopy analysis. The Phe_{X-D6} and Phe_{X-B5} pairs encoded multiply fluorinated Phe analogs with high fidelity (FIG. 6A). Estimated yield of sfGFP with Penta-fluoro Phe encoded by Phe_{X-D6} was ˜34 μg per gram of HEK cells. From the E. coli permissivity evaluation we note a primary feature distinguishing Phe_{X-B5} from Phe_{X-D6} is the ability of the former to recognize 2-mono fluoro Phenylalanine. However, in HEKT cell expression of sfGFP-N150TAG with Phe_{X-B5} and 2-mono fluoro Phe, we observed evidence of widespread encoding of 2-mono fluoro Phe at Phe-codons. This observation suggests that the endogenous Phe RS/tRNA pair cannot effectively distinguish between 2-mono F Phe and Phe, preventing site-specific encoding of 2-monofluoro Phe in HEKT cells and likely other human cells as well. Overall, the ESI-mass spectroscopy analysis of the pure ncAA-protein demonstrate the fidelity of the Phe_{X} RS group to encode 6 of the fluorinated ncAAs; 2 di-fluorinated (2,5/2,6), a tri-fluorinated (2,3,6), two tetra-flourinated (2,3,5,6/2,3,4,5), and the penta-fluorinated phenylalanine in HEKT cells with high efficiency and fidelity. Dose-response experiments with ascending concentrations of the ncAAs indicate that a few combinations (such as Phe_{X-D6}+penta-fluoro Phe) are useable at sub-millimolar concentrations of ncAA but that, in general, 1-2 mM is necessary for efficient ncAA-protein expression (FIG. 6B).

### Use of Pheₓ Synthetases to Site-Specifically Encode Fluoro-Phe ncAAs into Human Proteins

Finally, we demonstrate the utility of the tRNA/Pheₓ for fluoro-Phe ncAA encoding within two large, human ion channels of high clinical significance. Mutations within the ˜150 kDa Cystic Fibrosis Transmembrane Conductance Regulator (hCFTR) cause the life-shortening disease Cystic Fibrosis (CF) (FIG. 7A), while mutation and dysregulation of the ˜200 kDa cardiac sodium channel (hNa_{V} 1.5) are associated with various cardiovascular diseases including cardiac arrythmia and heart failure (FIG. 7B). These proteins have been studied in a wide variety of eukaryotic cell hosts, but mammalian cell expression is either essential or preferred for many types of functional, biochemical, and structural analyses of channel regulation or pharmacology. We introduced TAG codons at position F508 in CFTR and at F1486 in hNav 1.5 and co-transfected these expression plasmids with Phe_{X-D6} in the presence or absence of 2,3,6F Phe. We harvested the cells two days post transfection and assessed expression via western blot of cell lysates. In both cases, expression of full-length channels (as indicated by antibodies specific for C-terminal epitopes) was dependent on addition of 2,3,6F Phe in the growth media (FIGS. 7A-7C), demonstrating specific encoding by Phe_{X-D6}. Independent confirmation of 2,3,6F Phe encoding within Nav 1.5 was accomplished via expression of Nav 1.5 F1486 (2,3,6F Phe) via PheX-D6, purification via FLAG TAG and in-gel tryptic digestion/MS-MS determination (FIG. 7D). Finally, WT and Nav 1.5 F1486(2,3,6F Phe) channels were expressed in HEK cells and recorded via the whole cell patch clamp configuration. In agreement with the western blot results, robust macroscopic currents were observed for both WT and mutant channels; these channels appeared to activate and inactivate normally in response to membrane depolarization (FIG. 7E). Analysis of gating parameters revealed that encoding 2,3,6F-Phe at position F1486 caused a subtle enhancement of channel inactivation. This was apparent in a small (˜7 mV) but statistically significant left shift in the midpoint of steady state inactivation (FIG. 7G, 7H). Interestingly, data from exhaustive natural mutation of this critical phenylalanine previously revealed a strong correlation between hydrophobicity and efficiency of inactivation; no natural mutation fully reproduced WT (Phe) behavior. The data here suggest that the interaction of the IFM particle for its inactivated-state receptor is enhanced via near-isosteric fluorine substitution on the benzene ring of F1486. This is consistent with studies of model hydrophobic cores wherein targeted fluorination enhanced thermodynamic stability over native levels.

### Discussion

The electrostatic contributions of aromatic residues to protein-protein interactions and ligand recognition are becoming increasingly appreciated, but available tools limit the ability to test proposed mechanistic hypotheses. The tRNA/Phe_{X}-RS pairs reported herein allow for the encoding of a wide spectrum of fluorinated phenylalanine residues, which can be used to tune the electrostatic contributions of the aromatic side chain in pi-pi and cation-pi interactions (FIGS. 1A-1B, 3C). The majority of the fluorinated phenylalanine residues used in this study are commercially available, making this tool set immediately adoptable for those studying aromatic side change contributions in prokaryotes and eukaryotes. As these tRNA/RS pairs were first evolved for a larger amino acid surrogate, para-methyl-Phe, it is not uprising that the more highly fluorinated species which take up a slightly larger volume tend to encode most efficiently into proteins (FIGS. 3A-3B, 5A-5B). We view the fact that penta-fluoro and tetra-fluoro phenylalanine analogs encode so well as fortuitous because it enables the investigator to gain valuable early information on the electrostatic role Phe residues given the strong electrostatic perturbation imparted by the fluorines. For example, penta-fluoro phenylalanine is estimated to bear only ˜12-34% of the cation-pi binding potential of native Phe (FIG. 3C), whereas tri-fluorinated phenylalanine bears ˜40-60%, depending on the cation involved.

The efficiency of the synthetases as demonstrated herein suggest that they will be useful for the site-specific encoding of non-canonical phenylalanine analogs within proteins in E. coli and HEK cells at levels sufficient for biochemical characterization. In HEK cells, the yield of the most robust combination (Phe_{X-D6} and penta-fluoro phenylalanine) was 34 μg per gram of cell pellet using transient transfection. This is comfortably within the range necessary for structure determination by cryo-EM, provided the protein is efficiently purified and the growth is sufficiently scaled. The system may be further optimized via construction of synthetase-incorporated baculoviruses or stable cell lines. The function of the synthetases in mammalian cells enables their use to answer questions that were previously intractable with other systems (prokaryotic, cell-free, Xenopus oocyte) and in principle can be extended to express and characterize myriad membrane and soluble proteins. Beyond mechanistic work, the synthetases may be used to site-specifically incorporate fluoro-Phenylalanine NMR probes, and an optimized system utilizing these synthetases may enable the biotechnological production of therapeutic proteins wherein fluorinated phenylalanine residues are encoded for pharmacologically advantageous reasons.

### Methods

### Sources of Amino Acids

Synthesis of 2,3,5,6 tetrafluoro-paramethyl-Phe: 4 methyl Phe was carried out according to the report of Mikaye-Stoner et al. (Miyake-Stoner, S. J., et al., Generating permissive site-specific unnatural aminoacyl-tRNA synthetases. Biochemistry, 2010. 49(8): p. 1667-77). Mass spectrometry revealed mass of 252.05 in positive mode and 250.06 in negative mode, which agreed with the predicted mass of 251.06.

Synthesis of 2,3,5,6 tetrafluoro Phe: All solvents and reagents were supplied by Sigma Aldrich and were used as-is unless explicitly stated. Dry nitrogen was supplied by Praxair and passed through two moisture scrubbing columns of dry calcium sulfate (Drierite) prior to use. HPLC analyses were performed on a Waters 1525 Binary HPLC pump equipped with a Waters 2998 Photodiode Array Detector, employing Sunfire C18 analytical (3.5 μm, 4.6 mm×150 mm, 0.8 ml/min) or preparative (5.0 μm, 19 mm×150 mm, 10 ml/min) columns and Empower software. Buffers were drawn in linear gradients from 100% A (50 mM ammonium acetate) to 100% B (acetonitrile) over 30 min. Mass spectra were recorded on a Waters QToF Premier Quadrupole instrument, in both positive and negative modes. The amino acid was synthesized according to a published procedure (Zheng, H., K. Comeforo, and J. Gao, Expanding the fluorous arsenal: tetrafluorinated phenylalanines for protein design. J Am Chem Soc, 2009. 131(1): p. 18-9) with the following small modification: in lieu of ethanol extraction, following saponification of the methyl amide, the neutralized and lyophilized salt was purified directly via HPLC. The product was lyophilized to a white powder. (Calculated mass for C₉H₇F₄NO₂: 237.1 Da, found 238.0 Da (M+1)/236.0 Da (M−1) for MS in positive and negative modes).

The other Phe analogs were acquired commercially as follows: 2F Phe-Astatech, cat #73308; 4F Phe-Chem-impex cat #02572; 2,4F Phe-1ClickChemistry, cat #5C96757; 2,5F Phe-Astatech, cat #60350; 2,6F-Phe HCl salt-Chem-impex, cat #24171; 3,5F Phe Chem-impex, cat #04123; 2,3,6F-Phe-Astatech, A50355; 3,4,5F Phe-Chem-impex, cat #07394; 2,3,4,5F Phe-Enamine, cat #en300-27751009; 2,3,4,5,6F Phe-Chem-impex, cat #07183.

### PylRS Library Screen in E. coli

A plasmid library composed of the 20 canonical amino acids randomized at sites Asn311, Cys313, Val366, Trp382, and Gly386 was used alongside positive (pREP-pylT) and negative selection (pYOBB2-pylT) plasmids to select for aminoacyl-tRNA synthetases unique to pMe-2,3,5,6-tetrafluoro-Phe. All incubations for selection are carried out at 37° C. for 16 hours unless otherwise noted. All recovery steps last 1 hour and are shaken at 250 rpm. Cultures are grown at 37° C. for 16 hours and shaken at 250 rpm.

For positive selection, 500 μL of DH10B electrocompetent cells containing the positive pREP-pylT selection plasmid were transformed by electroporation with 1 μg of library plasmid. Cells were recovered in SOC media at 37° C. for 1 hour. Recovered cells were serially plated from 10 to 10⁻⁶, and grown for 16 hours at 37° C. to insure proper library coverage. Coverage was calculated to ˜150×, representing >99% of possible library members. The pooled recovery was used to inoculate 500 mL of LB media with 50 μg/mL of Kanamycin (Kan) and 25 μg/mL of tetracycline (Tet). The culture was grown overnight to saturation. This saturated culture was used to inoculate 500 mL of fresh LB media. This was grown to an OD₆₀₀ of 4.1, and 100 μL was plated on each of ten 15 cm LB-agar plates containing 50 μg/mL Kan, 25 μg/mL Tet, 40 μg/mL chloramphenicol (Cm), and 1 mM ncAA. Plates were incubated overnight. To harvest the resulting cells, 5 mL of LB-media was added to each plate. Colonies were then scraped from plates, pooled, and recovered for 1 hour. Cells were then pelleted and plasmid DNA extracted using a Macherey-Nagel miniprep kit. Resulting DNA was then plasmid separated by isolating the library plasmid on a 0.8% agarose gel vis agarose gel electrophoresis and extracting using the Thermo Scientific GeneJET gel extraction kit.

For negative selection, 100 ng of plasmid DNA from the positive selection was transformed into 50 μL of DH10B cells containing PYOBB2-pylT using electroporation and recovered in 1 mL SOC media. Following recovery, 100 μL was plated on each of 3 LB-agar plates containing 50 μg/mL Kan, 25 μg/mL Cm, and 0.2% arabinose. After 16 hours, cells were scraped and DNA prepped as described above.

To identify functional synthetase variants, 100 ng of the pBK DNA from negative selection was transformed into 25 μL of electrocompetent DH10B cells containing the reporter plasmid, pALS-plyT. This plasmid contains the sfGFP reporter with a TAG site at residue 150. In the presence of the selected ncAA, the TAG site will be suppressed, and the resulting colonies should appear green. Transformed cells were recovered in 1 mL SOC media. After recovery, cells were diluted by a factor of 100, and 100 μL of this dilution was plated on three 15 cm auto-inducing plates. All plates contain 50 μg/mL Kan and 25 μg/mL Tet; two contain 1 mM ncAA and the other is left without ncAA as a control. Plates were incubated overnight. After 16 hours, plates were kept at RT for 24 hours to further develop. After fully maturated, colonies that appeared green were individually selected and used to inoculate 500 μL of non-inducing media containing 50 μg/mL Kan and 25 μg/mL Tet in a 96-well block. This block was incubated for 20 hours at 37° C. shaking at 300 rpm. After adequate growth, 20 μL of each well was used to inoculate two 96-well blocks with 500 μL of auto-inducing media with 50 μg/mL Kan and 25 μg/mL Tet. One block contained 1 mM ncAA while the other did not. After 24 hours of incubation (37° C., 300 rpm), sfGFP fluorescence was measured. The 30 highest performing synthetases were sequenced, and 17 unique sequences were identified.

### UP50 Measurements

Electrocompetent DH10B cells containing the pALS reporter plasmid were transformed with isolated pBK plasmids containing the Phe_{X} synthetases. The transformants of 8 synthetases, A11, A12, B5, B6, C4, C10, D6 and D7, were used to inoculate five 500 μL cultures each, consisting of auto-induction media with 50 μg/mL Kan and 25 μg/mL Tet containing no ncAA, 0.1 mM, 0.2 mM, 0.5 mM, or 1 mM ncAA. These were performed in duplicate, and fluorescence and OD₆₀₀ were measured after 24 hours.

### Permissivity Screen (E. coli)

Electrocompetent DH10B cells containing the pALS reporter plasmid were transformed with isolated pBK plasmids containing the synthetases. Cultures of 5 mL were inoculated with single colonies of each synthetase in non-inducing media with 50 μg/mL kanamycin and 25 μg/mL tetracycline and grown for 16 hours. Noncanonical amino acid stocks were made at 100× their final concentration. 96-well blocks were prepared with 500 μL of auto-induction media containing antibiotics, and ncAAs with a final working concentration of 1 mM were added to their respective wells. Each well was then inoculated with a corresponding synthetase, yielding a final 96-well block where each synthetase was tested against each amino acid of interest. Cultures were grown in duplicate and fluorescence measurements were taken at 24 hours.

### Expression and Purification of sfGFP (E. coli)

Previously existing E. coli containing sfGFP150TAG and a given Rs in non-inducing media were used to inoculate 5 mL cultures of non-inducing media containing 50 μg/mL Kan and 25 μg/mL Tet. Cultures were incubated for 20 hours at 37° C., shaking at 250 rpm. After adequate growth, 500 μL of the 5 mL cultures were used to inoculate 50 mL cultures of auto-inducing media containing 1 mM ncAA. Cultures were incubated for 24 hours (37° C., 250 rpm), after which cultures were then spun down at 5000 rcf for 10 minutes and resuspended in 10 mL of a Tris buffer solution containing 100 mM Tris, 0.5 M NaCl, and 5 mM imidazole. Cells were lysed by micro-fluidization at 18,000 psi and centrifuged at 20,0000 rcf for 30 minutes. sfGFP proteins in cell lysate were bound to TALON Metal Affinity Resin (bed volume of 100 μL) at 4° C. for 1 hour. Lysate and bound resin were transferred to a gravity-flow column and the resin was washed with 30 bed volumes of buffer. Protein was eluted using Tris buffer solution previously described supplemented with 200 mM imidazole. Protein concentration was assessed by comparing sfGFP fluorescence to a standard curve and submitted for ESI-MS.

As is apparent in the traces shown in FIG. 4, the deconvoluted mass spectra showed multiple masses for WT as well as mutant sfGFP. The spectra were analyzed in-house by Novatia (Promass) to yield relative intensities at given masses. In addition to the base (main) peak, both WT and mutant sfGFP displayed minor masses of ˜−131 Da (lacking Methionine 1), ˜−1378 Da, and ˜−4165 Da. For the purpose of estimating encoding fidelity, these were considered part of the expected mass, and all other signals were considered non-expected mass. The one exception is a +36.9 Da peak in the spectra of Phe_{X-D6}+2,3,6F Phe which was classified as a K+ adduct of the expected mass by Promass software. For Phe_{X-D6}, encoding fidelity for Penta-, 2,3,5,6-, 2,3,6- and 2,6-flouro Phe was 98.2%, 98.7%, 96.7%, and 95.0% respectively. For Phe_{X-B5}, encoding fidelity for Penta-, 2,3,5,6-, 2,3,6-, 2,6-, and 2-fluoro Phe was 97.5%, 97.9%, 88.1%, 81.8%, and 95.6%.

### Mammalian Cell Culture

HEK 293T cells (CRL 3216) were maintained in DMEM high glucose medium supplemented with Pen/Strep, L glutamine, and 10% FBS (Sigma). Cells were used at passages 5 to 35.

### Construction of pAcBac1-Pheₓ Plasmid

The pAcBac1.tR4-MbPyl, used to express a MbPylRS in mammalian cells, was a gift from Peter Schultz (Addgene plasmid #50832). A WT human codon optimized version of the MbPylRS was substituted into the pAcBac1.tR4-MbPyl and was a gift from Jason Chin. The active site sequence with mutated residues was synthesized as a gBlock from Integrated DNA Technologies. Relevant DNA fragments were assembled using NEBuilder HiFi DNA Assembly Master Mix, transformed and isolated from NEB Stable cells, and sequence confirmed.

### Permissivity Screen (Mammalian)

HEKT cells were seeded in 60 mm dishes so that they would be approximately 60-80% confluent the next day. We transfected the cells using 1.75 μg of the PacBac1-based synthetase plasmid (Phe_{X-D6} or Phe_{X-B5}) and 0.75 μg of sfGFP_N150TAG per dish. Polyjet was used (7.5 μl per dish, 2.5 mL media volume). Media was exchanged to contain ncAAs at a given concentration before transfection and approximately 16 hours after transfection. Amino acids were solubilized either in NaOH for those synthesized as free acids (penta-flouro, 2,3,6 trifluoro, 3,4,5 trifluoro, 2,5 difluoro, 2,4 difluoro, 2 mono) or in HCl for those synthesized as HCl salts (2,3,5,6 tetrafluoro, 2,3,4,5 tetrafluoro, 2,6 difluoro). Cells were imaged and harvested approximately 24 hrs after transfection. Images were taken with 20× objective using a Leica DFC9000GT camera with 700 ms exposure, without binning. Excitation was through an X-Cite 120 LED (Lumen Dynamics) set to 100% intensity. For bright field images, light was adjusted manually. To harvest, cells were washed twice with ice cold DPBS, then sloughed off the dishes in 1 mL ice cold DPBS supplemented with Roche protease inhibitors. Cells were collected in microcentrifuge tubes and pelleted by centrifugation. Supernatant was removed and the cell pellets were flash frozen in liquid nitrogen, then stored at −80 C. To lyse, 350 ul of RIPA buffer (Sigma) plus Roche protease inhibitor tablets was added to each pellet. After cell debris and nuclei were cleared via centrifugation, GFP fluorescence was read from supernatants in 96 well plates; for each concentration of amino acid, at least 6 readings were made from at least 2 transfections. The level of fluorescence from lysates from untransfected cells averaged 21.7±2.1 RFU. By comparison, cells transfected with sfGFP_N150TAG and Phe_{X-D6} averaged 26.9±5.6 RFU and Phe_{X-B5} averaged 43.1±2.5. The maximum background signal thus attributable to Phe_{X-B5} and Phe_{X-D6} was thus ˜5 RFU and ˜21.4 RFU respectively.

### Expression for Purification and Mass Spectrometry (Mammalian)

HEK 293T cells were expanded in 10 mm dishes. For a given synthetase/amino acid combination, the size of the transfection (number of plates) was estimated from differences in relative sfGFP_N150TAG yield in the multi-specificity screens, with poorer encoders requiring more cell mass. Overall, 7-40 dishes were transfected with master mixes such that each dish received 3.5 μg of pACBAC1, 1.5 μg sfGFP_N150TAG, and 15 μL of PolyJET according to manufacturer's instructions. Media was changed before transfection and approximately 18 hrs after transfection. Cells were harvested approximately 36 hours after transfection. To harvest, each dish was washed twice with DPBS. Cells were harvested via scraping in ice-cold DPBS plus Roche protease inhibitors, on ice, pelleted by centrifugation, and flash frozen in liquid nitrogen. Cells were lysed in hypotonic lysis buffer (10 mM Tris/HCl pH 8, Roche protease inhibitors, 0.1% Triton X 100 Sigma) via dounce homogenization on ice. Cell debris and nuclei were cleared via centrifugation. Clarified lysate was diluted 1:2 in Wash buffer (25 mM Tris/HCl pH 8, 20 mM Imidazole, 150 mM NaCl) and applied to pre-equilibrated Ni-NTA resin (Qiagen). Column was washed in at least 30 volumes of wash buffer, then protein was eluted in elution buffer (25 mM Tris/HCl pH 8, 250 mM Imidazole, 150 mM NaCl). Elution was exchanged into 100 mM TEAB, prepared via 1:10 dilution of 1 M Stock (thermofisher) in ultrapure water. Finally, protein was concentrated to ˜0.5 mg/ml, flash frozen, and submitted to Novatia Inc for ESI mass spec of the intact protein. The LC-UV traces from Novatia indicated that purified WT and mutant sfGFP existed as three predominant isoforms, a dominant peak and two minor peaks representing +532+/−1 Da and −42+/−1 Da mass differences. Note that the WT condition was co-transfected with Phe_{X-B5} synthetase, which did not affect the mass (within <1 Da of the same WT sfGFP expressed alone). As was also the case when expressed in E. coli, the deconvoluted mass spectra for the dominant LC peak showed multiple masses for WT, as well as mutant sfGFP, from HEK cells. The spectra was analyzed in-house by Novatia (Promass software) to yield relative intensities of the dominant mass and any other apparent peaks. WT and mutant sfGFP shared minor peaks of ˜−42 Da (appx 2% of integration), ˜−1288 Da (appx 2% of integration) ˜183 Da (appx 3% of integration), and ˜+53 Da (appx 1% of integration) in common and thus, for the sake of estimation of encoding fidelity, these were counted among the expected mass. To be conservative, all other peaks were considered unexpected mass, even though they may not represent actual misincorporation. Fidelity for Phe_{X-D6} and Penta-, 2,3,4,5-, 2,3,5,6-, 2,3,6-, 2,6-, and 2,5-fluoro phenylalanine were estimated to be 97.5%, 100%, 98.2%, 98.5%, 97.6%, and 95.6%. For Phe_{X-B5}, fidelity was 96.2%, 97.4%, 100%, 100%, 100%, and 91.3% for the same species.

To accurately estimate sfGFP yield in a scenario wherein loss during purification is minimized, we transfected and harvested 31-10 cm dishes of HEKT cells with Phe_{X-D6} and sfGFP_N150TAG in the presence of 2 mM penta-fluoro Phenylalanine. sfGFP was purified as described above. Percent sfGFP was quantified using band detection and densitometry in Biorad Imagelab software.

### Expression of Channel Constructs with Site-Specific Encoding of 2,3,6F Phe

pCMV-CFTR was a gift from Paul McCray (University of Iowa). WT CFTR-TAA (the existing TAG stop codon changed to TAA), F508 TAG CFTR (TAG amber codon introduced at F508 in the above WT construct), as well as WT and F1486TAG hNav 1.5_strep_HIS_strep were produced using standard methods and sequenced through the open reading frame. For expression of WT CFTR, 10 cm dishes of HEK 293T cells were transfected with 1.5 μg WT CFTR-TAA and 0.25 μg WT GFP. For expression of WT hNav 1.5, 10 cm dishes of HEK 293T cells were transfected with 1.5 μg of WT hNav 1.5_strep_HIS_strep and 0.25 μg WT GFP. For encoding of 2,3,6F Phe, 10 cm dishes of HEK 293T cells were transfected from master mixes of DNA such that each dish received 3.5 μg of Phe_{X-D6} and 0.25 μg of sfSFGFP_N150TAG. Depending on condition, 2 μg of F508TAG-CFTR or 1.5 μg of F1486TAG-Nav 1.5-SHS were added. Media was changed twice (˜16 hr and ˜32 hr post transfection), and cells were harvested ˜44 hrs post transfection and flash frozen in aliquots. Cell pellets were resuspended in RIPA (Sigma Aldrich) plus Roche protease inhibitors. 60 μg of protein from cleared, unconcentrated lysate (or a dilution) was loaded on 4-20% SDS gels. For Western blotting, gels were transferred to Nitrocelllose membranes and probed with anti-CFTR (ab596 from Cystic Fibrosis Foundation, 1:1750) or anti-Nav 1.5 (D9J7S, Cell Signaling, 1:5000) overnight in 5% milk in TBST. Blots were washed, probed with HRP secondaries (1:10,000) and imaged using Clarity ECL (Biorad). Blots were subsequently probed for beta-actin using HRP-conjugated primary (AC-15, Novus Biologicals 1:2000) appx 3 hours in 5% milk in TBST. Blots were washed and imaged using Clarity ECL (Biorad). Densitometry (background subtracted integrated density) was done in ImageJ.

For patch clamp recording of hNaᵥ 1.5, in parallel we co-transfected 10 cm dishes with 3.5 μg of Phe 6, 0.25 μg of sfGFP_N150TAG, and 1.5 μg of either WT hNaᵥ 1.5 strep_HIS_strep or hNaᵥ 1.5 F1486TAG strep_HIS_strep. Both conditions were cultured in 2 mM 2,3,6 Trifluoro Phe throughout. Media was changed at 16 hrs and again when seeding onto 35 mm dishes for recording. Recordings were done approximately ˜42 hrs post transfection. Pipette solution contained 105 CsF, 35 NaCl, 10 EGTA, and 10 Hepes (pH 7.4). The bath contained (in mmol/L) 150 NaCl, 2 KCl, 1.5 CaCl2, 1 MgCl2, and 10 Hepes (pH 7.4). Pipette resistances were around 2 mΩ and series resistance was compensated ≥85%. For generation of I/V curves, cells were held at −140 mV and pulsed in 5 mV depolarizing increments. For measurement of steady state inactivation, cells were held at −140 mV and conditioned for 500 ms in 5 mV depolarizing increments. Test ulse was at −30 mV. Normalized G/V and SSI curves were fit to Boltzmann functions using Origin software.

For purification of hNav 1.5 F1486 (2,3,6F Phe), ten 10 cm dishes of HEKT cells were co transfected so that each dish received 1.5 μg pCDNA hNav 1.5 F1486TAG_FLAG, 3.5 μg PheX-D6, and 0.25 μg sfGFPN150TAG plasmids. Media changes were as described for the western blots. Channels were solubilized and purified as described in the report of the cryo-EM structure of the cardiac channel [47], with the following modifications. First, we used DDM/CHS as detergent throughout rather than exchanging to GDN during washes. Secondly, the elution was concentrated and run directly on SDS page gel instead of being subjected to size exclusion chromatography. Gel band excision, tryptic digestion, and MS/MS were performed by the Iowa Proteomics Core using standard methods.

### Quantum Calculations of Cation Pi Binding Potential

All the models are prepared using GaussView 6 and all quantum chemical calculations are performed using Gaussian 16. Full geometry optimizations are carried out at M06/6-31G(d,p) level of theory. In some fluorinated aromatic cases where the optimization resulted in geometries that are not considered cation-π interactions, the binding energy is obtained using the single-point method. In this approach full geometry optimization of the cation and the aromatic system is performed without the fluorination at the position which caused incompatible geometries upon optimization. Then fluorine is appended to the aromatic ring with a bond distance calculated from the aromatic system with fluorination at the desired position which is optimized in isolation. The energy of this system is calculated and used to find the binding energy in the single-point calculations.

### EXAMPLE 2

Evidence from the small peptide field (model protein cores) has established that the substitution of specific phenylalanine residues with fluorinated phenylalanine analogs increases thermodynamic stability above native levels. A scalable method for producing recombinant proteins bearing site-specific substitutions of a wide variety of these unnatural amino acids has been developed and validated. Specifically, novel aminoacyl-tRNA synthetase enzymes were identified that recognize fluorinated analogs of phenylalanine and catalyze their site-specific encoding into proteins in prokaryotic and eukaryotic cells.

Using molecular and chemical biology methods to optimize the pharmacological profile of recombinant protein therapeutics is traditionally limited to design with the “canonical” amino acids. The present approach enables a subtle but chemically significant change in the design of injectable protein therapeutics that is expected to result (depending on intention and specific use) in supra-physiological stability, enhanced specificity of physiological interaction, and resistance to proteolytic digestion.

Exemplary novel enzymes useful in this approach are provided below:

Phe_{x-D6} ECOLI
(SEQ ID NO: 1)
MDKKPLDVLISATGLWMSRTGTLHKIKHHEVSRSKIYIEMACGDHLVV
NNSRSCRTARAFRHHKYRKTCKRCRVSDEDINNFLTRSTESKNSVKVR
VVSAPKVKKAMPKSVSRAPKPLENSVSAKASTNTSRSVPSPAKSTPNS
SVPASAPAPSLTRSQLDRVEALLSPEDKISLNMAKPFRELEPELVTRR
KNDFQRLYTNDREDYLGKLERDITKFFVDRGFLEIKSPILIPAEYVER
MGINNDTELSKQIFRVDKNLCLRPMLAPTLYNYLRKLDRILPGPIKIF
EVGPCYRKESDGKEHLEEFTMVAFMQMGSGCTRENLEALIKEFLDYLE
IDFEIVGDSCMVYGDTLDIMHGDLELSSAGVGPVSLDREWGIDKPTIG
AGFGLERLLKVMHGFKNIKRASRSESYYNGISTNL
Phe_{x-D6} HEK
(SEQ ID NO: 2)
MACPVPLQLPPLERLTLDDYKDDDDKDKKPLDVLISATGLWMSRTGTL
HKIKHHEVSRSKIYIEMACGDHLVVNNSRSCRTARAFRHHKYRKTCKR
CRVSDEDINNFLTRSTESKNSVKVRVVSAPKVKKAMPKSVSRAPKPLE
NSVSAKASTNTSRSVPSPAKSTPNSSVPASAPAPSLTRSQLDRVEALL
SPEDKISLNMAKPFRELEPELVTRRKNDFQRLYTNDREDYLGKLERDI
TKFFVDRGFLEIKSPILIPAEYVERMGINNDTELSKQIFRVDKNLCLR
PMLAPTLYNYLRKLDRILPGPIKIFEVGPCYRKESDGKEHLEEFTMVA
FMQMGSGCTRENLEALIKEFLDYLEIDFEIVGDSCMVYGDTLDIMHGD
LELSSAGVGPVSLDREWGIDKPTIGAGFGLERLLKVMHGFKNIKRASR
SESYYNGISTNL
Phe_{x-B5} ECOLI
(SEQ ID NO: 3)
MDKKPLDVLISATGLWMSRTGTLHKIKHHEVSRSKIYIEMACGDHLVV
NNSRSCRTARAFRHHKYRKTCKRCRVSDEDINNFLTRSTESKNSVKVR
VVSAPKVKKAMPKSVSRAPKPLENSVSAKASTNTSRSVPSPAKSTPNS
SVPASAPAPSLTRSQLDRVEALLSPEDKISLNMAKPFRELEPELVTRR
KNDFQRLYTNDREDYLGKLERDITKFFVDRGFLEIKSPILIPAEYVER
MGINNDTELSKQIFRVDKNLCLRPMLAPTLYNYLRKLDRILPGPIKIF
EVGPCYRKESDGKEHLEEFTMVGFAQMGSGCTRENLEALIKEFLDYLE
IDFEIVGDSCMVYGDTLDIMHGDLELSSAVVGPVSLDREWGIDKPWIG
AGFGLERLLKVMHGFKNIKRASRSESYYNGISTNL
Phe_{x-B5} HEK
(SEQ ID NO: 4)
MACPVPLQLPPLERLTLDDYKDDDDKDKKPLDVLISATGLWMSRTGTL
HKIKHHEVSRSKIYIEMACGDHLVVNNSRSCRTARAFRHHKYRKTCKR
CRVSDEDINNFLTRSTESKNSVKVRVVSAPKVKKAMPKSVSRAPKPLE
NSVSAKASTNTSRSVPSPAKSTPNSSVPASAPAPSLTRSQLDRVEALL
SPEDKISLNMAKPFRELEPELVTRRKNDFQRLYTNDREDYLGKLERDI
TKFFVDRGFLEIKSPILIPAEYVERMGINNDTELSKQIFRVDKNLCLR
PMLAPTLYNYLRKLDRILPGPIKIFEVGPCYRKESDGKEHLEEFTMVG
FAQMGSGCTRENLEALIKEFLDYLEIDFEIVGDSCMVYGDTLDIMHGD
LELSSAVVGPVSLDREWGIDKPWIGAGFGLERLLKVMHGFKNIKRASR
SESYYNGISTNL
Phe_{x-A12} ECOLI
(SEQ ID NO: 5)
MDKKPLDVLISATGLWMSRTGTLHKIKHHEVSRSKIYIEMACGDHLVV
NNSRSCRTARAFRHHKYRKTCKRCRVSDEDINNFLTRSTESKNSVKVR
VVSAPKVKKAMPKSVSRAPKPLENSVSAKASTNTSRSVPSPAKSTPNS
SVPASAPAPSLTRSQLDRVEALLSPEDKISLNMAKPFRELEPELVTRR
KNDFQRLYTNDREDYLGKLERDITKFFVDRGFLEIKSPILIPAEYVER
MGINNDTELSKQIFRVDKNLCLRPMLAPTLYNYLRKLDRILPGPIKIF
EVGPCYRKESDGKEHLEEFTMVGFCQMGSGCTRENLEALIKEFLDYLE
IDFEIVGDSCMVYGDTLDIMHGDLELSSAAVGPVSLDREWGIDKPWIG
AGFGLERLLKVMHGFKNIKRASRSESYYNGISTNL
Phe_{x-A12} HEK
(SEQ ID NO: 6)
MACPVPLQLPPLERLTLDDYKDDDDKDKKPLDVLISATGLWMSRTGTL
HKIKHHEVSRSKIYIEMACGDHLVVNNSRSCRTARAFRHHKYRKTCKR
CRVSDEDINNFLTRSTESKNSVKVRVVSAPKVKKAMPKSVSRAPKPLE
NSVSAKASTNTSRSVPSPAKSTPNSSVPASAPAPSLTRSQLDRVEALL
SPEDKISLNMAKPFRELEPELVTRRKNDFQRLYTNDREDYLGKLERDI
TKFFVDRGFLEIKSPILIPAEYVERMGINNDTELSKQIFRVDKNLCLR
PMLAPTLYNYLRKLDRILPGPIKIFEVGPCYRKESDGKEHLEEFTMVG
FCQMGSGCTRENLEALIKEFLDYLEIDFEIVGDSCMVYGDTLDIMHGD
LELSSAAVGPVSLDREWGIDKPWIGAGFGLERLLKVMHGFKNIKRASR
SESYYNGISTNL
Phe_{x-B6} ECOLI
(SEQ ID NO: 7)
MDKKPLDVLISATGLWMSRTGTLHKIKHHEVSRSKIYIEMACGDHLVV
NNSRSCRTARAFRHHKYRKTCKRCRVSDEDINNFLTRSTESKNSVKVR
VVSAPKVKKAMPKSVSRAPKPLENSVSAKASTNTSRSVPSPAKSTPNS
SVPASAPAPSLTRSQLDRVEALLSPEDKISLNMAKPFRELEPELVTRR
KNDFQRLYTNDREDYLGKLERDITKFFVDRGFLEIKSPILIPAEYVER
MGINNDTELSKQIFRVDKNLCLRPMLAPTLYNYLRKLDRILPGPIKIF
EVGPCYRKESDGKEHLEEFTMVAFMQMGSGCTRENLEALIKEFLDYLE
IDFEIVGDSCMVYGDTLDIMHGDLELSSAGVGPVSLDREWGIDKPTIG
AGFGLERLLKVMHGFKNIKRASRSESYYNGISTNL
Phe_{x-B6} HEK
(SEQ ID NO: 8)
MACPVPLQLPPLERLTLDDYKDDDDKDKKPLDVLISATGLWMSRTGTL
HKIKHHEVSRSKIYIEMACGDHLVVNNSRSCRTARAFRHHKYRKTCKR
CRVSDEDINNFLTRSTESKNSVKVRVVSAPKVKKAMPKSVSRAPKPLE
NSVSAKASTNTSRSVPSPAKSTPNSSVPASAPAPSLTRSQLDRVEALL
SPEDKISLNMAKPFRELEPELVTRRKNDFQRLYTNDREDYLGKLERDI
TKFFVDRGFLEIKSPILIPAEYVERMGINNDTELSKQIFRVDKNLCLR
PMLAPTLYNYLRKLDRILPGPIKIFEVGPCYRKESDGKEHLEEFTMVA
FMQMGSGCTRENLEALIKEFLDYLEIDFEIVGDSCMVYGDTLDIMHGD
LELSSAGVGPVSLDREWGIDKPTIGAGFGLERLLKVMHGFKNIKRASR
SESYYNGISTNL
Phe_{x-C4} ECOLI
(SEQ ID NO: 9)
MDKKPLDVLISATGLWMSRTGTLHKIKHHEVSRSKIYIEMACGDHLVV
NNSRSCRTARAFRHHKYRKTCKRCRVSDEDINNFLTRSTESKNSVKVR
VVSAPKVKKAMPKSVSRAPKPLENSVSAKASTNTSRSVPSPAKSTPNS
SVPASAPAPSLTRSQLDRVEALLSPEDKISLNMAKPFRELEPELVTRR
KNDFQRLYTNDREDYLGKLERDITKFFVDRGFLEIKSPILIPAEYVER
MGINNDTELSKQIFRVDKNLCLRPMLAPTLYNYLRKLDRILPGPIKIF
EVGPCYRKESDGKEHLEEFTMVQFSQMGSGCTRENLEALIKEFLDYLE
IDFEIVGDSCMVYGDTLDIMHGDLELSSAGVGPVSLDREWGIDKPTIG
AGFGLERLLKVMHGFKNIKRASRSESYYNGISTNL
Phe_{x-C4} HEK
(SEQ ID NO: 10)
MACPVPLQLPPLERLTLDDYKDDDDKDKKPLDVLISATGLWMSRTGTL
HKIKHHEVSRSKIYIEMACGDHLVVNNSRSCRTARAFRHHKYRKTCKR
CRVSDEDINNFLTRSTESKNSVKVRVVSAPKVKKAMPKSVSRAPKPLE
NSVSAKASTNTSRSVPSPAKSTPNSSVPASAPAPSLTRSQLDRVEALL
SPEDKISLNMAKPFRELEPELVTRRKNDFQRLYTNDREDYLGKLERDI
TKFFVDRGFLEIKSPILIPAEYVERMGINNDTELSKQIFRVDKNLCLR
PMLAPTLYNYLRKLDRILPGPIKIFEVGPCYRKESDGKEHLEEFTMVQ
FSQMGSGCTRENLEALIKEFLDYLEIDFEIVGDSCMVYGDTLDIMHGD
LELSSAGVGPVSLDREWGIDKPTIGAGFGLERLLKVMHGFKNIKRASR
SESYYNGISTNL
Phe_{x-D7} ECOLI
(SEQ ID NO: 11)
MDKKPLDVLISATGLWMSRTGTLHKIKHHEVSRSKIYIEMACGDHLVV
NNSRSCRTARAFRHHKYRKTCKRCRVSDEDINNFLTRSTESKNSVKVR
VVSAPKVKKAMPKSVSRAPKPLENSVSAKASTNTSRSVPSPAKSTPNS
SVPASAPAPSLTRSQLDRVEALLSPEDKISLNMAKPFRELEPELVTRR
KNDFQRLYTNDREDYLGKLERDITKFFVDRGFLEIRSPILIPAEYVER
MGINNDTELSKQIFRVDKNLCLRPMLAPTLYNYLRKLDRILPGPIKIF
EVGPCYRKESDGKEHLEEFTMVAFMQMGSGCTRENLEALIKEFLDYLE
IDFEIVGDSCMVYGDTLDIMHGDLELSSAGVGPVSLDREWGIDKPTIG
AGFGLERLLKVMHGFKNIKRASRSESYYNGISTNL
Phe_{x-D7} HEK
(SEQ ID NO: 12)
MACPVPLQLPPLERLTLDDYKDDDDKDKKPLDVLISATGLWMSRTGTL
HKIKHHEVSRSKIYIEMACGDHLVVNNSRSCRTARAFRHHKYRKTCKR
CRVSDEDINNFLTRSTESKNSVKVRVVSAPKVKKAMPKSVSRAPKPLE
NSVSAKASTNTSRSVPSPAKSTPNSSVPASAPAPSLTRSQLDRVEALL
SPEDKISLNMAKPFRELEPELVTRRKNDFQRLYTNDREDYLGKLERDI
TKFFVDRGFLEIRSPILIPAEYVERMGINNDTELSKQIFRVDKNLCLR
PMLAPTLYNYLRKLDRILPGPIKIFEVGPCYRKESDGKEHLEEFTMVA
FMQMGSGCTRENLEALIKEFLDYLEIDFEIVGDSCMVYGDTLDIMHGD
LELSSAGVGPVSLDREWGIDKPTIGAGFGLERLLKVMHGFKNIKRASR
SESYYNGISTNL
Phe_{xA11} ECOLI
(SEQ ID NO: 13)
MDKKPLDVLISATGLWMSRTGTLHKIKHHEVSRSKIYIEMACGDHLVV
NNSRSCRTARAFRHHKYRKTCKRCRVSDEDINNFLTRSTESKNSVKVR
VVSAPKVKKAMPKSVSRAPKPLENSVSAKASTNTSRSVPSPAKSTPNS
SVPASAPAPSLTRSQLDRVEALLSPEDKISLNMAKPFRELEPELVTRR
KNDFQRLYTNDREDYLGKLERDITKFFVDRGFLEIKSPILIPAEYVER
MGINNDTELSKQIFRVDKNLCLRPMLAPTLYNYLRKLDRILPGPIKIF
EVGPCYRKESDGKEHLEEFTMVAFMQMGSGCTRENLEALIKEFLDYLE
IDFEIVGDSCMVYGDTLDIMHGDLELSSANVGPVSLDREWGIDKPTIG
AGFGLERLLKVMHGFKNIKRASRSESYYNGISTNL
Phe_{xA11} HEK
(SEQ ID NO: 14)
MACPVPLQLPPLERLTLDDYKDDDDKDKKPLDVLISATGLWMSRTGTL
HKIKHHEVSRSKIYIEMACGDHLVVNNSRSCRTARAFRHHKYRKTCKR
CRVSDEDINNFLTRSTESKNSVKVRVVSAPKVKKAMPKSVSRAPKPLE
NSVSAKASTNTSRSVPSPAKSTPNSSVPASAPAPSLTRSQLDRVEALL
SPEDKISLNMAKPFRELEPELVTRRKNDFQRLYTNDREDYLGKLERDI
TKFFVDRGFLEIKSPILIPAEYVERMGINNDTELSKQIFRVDKNLCLR
PMLAPTLYNYLRKLDRILPGPIKIFEVGPCYRKESDGKEHLEEFTMVA
FMQMGSGCTRENLEALIKEFLDYLEIDFEIVGDSCMVYGDTLDIMHGD
LELSSANVGPVSLDREWGIDKPTIGAGFGLERLLKVMHGFKNIKRASR
SESYYNGISTNL

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A composition comprising a pyrrolysine-based aminoacyl-tRNA synthetase and a tRNA to form a pyrrolysine-based aminoacyl-tRNA synthetase/tRNA pair.

2. The composition of claim 1, wherein the pyrrolysine-based aminoacyl-tRNA synthetase is an E. coli compatible synthetase is selected from the group consisting of Phe_{X-B5} (SEQ ID NO:3), Phe_{X-D6} (SEQ ID NO:1), Phe_{X-A12} (SEQ ID NO:5), Phe_{X-B6} (SEQ ID NO:7), Phe_{X-C4} (SEQ ID NO:9), Phe_{X-D7} (SEQ ID NO:11), and Phe_{X-A11} (SEQ ID NO:13).

3. The composition of claim 1, wherein the pyrrolysine-based aminoacyl-tRNA synthetase is a mammalian compatible synthetase is selected from the group consisting of Phe_{X-B5} (SEQ ID NO:4), Phe_{X-D6} (SEQ ID NO:2), Phe_{X-A12} (SEQ ID NO: 6), Phe_{X-B6} (SEQ ID NO:8), Phe_{X-C4} (SEQ ID NO:10), Phe_{X-D7} (SEQ ID NO:12), and Phe_{X-A11} (SEQ ID NO:12).

4. The composition of any one of claims 1-3, wherein the tRNA is pyrollysine tRNA (PylT) from Methanogenic archaea:
(SEQ ID NO: 17)
GGAAACCUGAUCAUGUAGAUCGAACGGACUCUAAAUCCGUUCAGCCGG
GUUAGAUUCCCGGGGUUUCCGCCA
or
(SEQ ID NO: 18)
GGAAACCUGAUCAUGUAGAUCGAACGGACUCUAAAUCCGUUCAGCCGG
GUUAGAUUCCCGGGGUUUCCGCCA.

5. The composition of any one of claims 1-4, further comprising a non-canonical amino acid (ncAA).

6. The composition of claim 5, wherein the ncAA is selected from the group consisting of 2-mono fluoro Phenylalanine, 2,6 di-fluoro Phenylalanine, 2,3,6 tri-fluoro Phenylalanine, 2,3,5,6 tetra-fluoro Phenylalanine, 2,3,4,5 tetra-fluoro Phenylalanine, 2,3,4,5,6 penta-fluoro Phenylalanine, para-methyl-tetrafluoro Phenylalanine, para-methyl-2-mono fluoro Phenylalanine, and para-methyl-2,3-di-fluoro Phenylalanine.

7. A composition comprising a cell comprising synthetase Phe_{X-B5} (SEQ ID NO:3), Phe_{X-D6} (SEQ ID NO:1), Phe_{X-A12} (SEQ ID NO:5), Phe_{X-B6} (SEQ ID NO:7), Phe_{X-C4} (SEQ ID NO:9), Phe_{X-D7} (SEQ ID NO:11), Phe_{X-A11} (SEQ ID NO:13), Phe_{X-B5} (SEQ ID NO: 4), Phe_{X-D6} (SEQ ID NO:2), Phe_{X-A12} (SEQ ID NO:6), Phe_{X-B6} (SEQ ID NO:8), Phe_{X-C4} (SEQ ID NO:10), Phe_{X-D7} (SEQ ID NO:12), or Phe_{X-A11} (SEQ ID NO:12).

8. The composition of claim 7, wherein the cell is a prokaryotic cell, and the synthetase is Phe_{X-B5} (SEQ ID NO:3), Phe_{X-D6} (SEQ ID NO:1), Phe_{X-A12} (SEQ ID NO:5), Phe_{X-B6} (SEQ ID NO:7), Phe_{X-C4} (SEQ ID NO:9), Phe_{X-D7} (SEQ ID NO:11), or Phe_{X-A11} (SEQ ID NO: 13).

9. The composition of claim 7, wherein the cell is a eukaryotic cell, and the synthetase is Phe_{X-B5} (SEQ ID NO:4), Phe_{X-D6} (SEQ ID NO:2), Phe_{X-A12} (SEQ ID NO:6), Phe_{X-B6} (SEQ ID NO:8), Phe_{X-C4} (SEQ ID NO:10), Phe_{X-D7} (SEQ ID NO:12), or Phe_{X-A11} (SEQ ID NO:12).

10. A composition comprising a cell free system comprising a pyrrolysine-based aminoacyl-tRNA synthetase and a tRNA to form a pyrrolysine-based aminoacyl-tRNA synthetase/tRNA pair.

11. A method of site-specific encoding of a fluorinated phenylalanine amino acid in a cell comprising co-transfecting the cell with synthetase Phe_{X-B5} (SEQ ID NO:3), Phe_{X-D6} (SEQ ID NO:1), Phe_{X-A12} (SEQ ID NO:5), Phe_{X-B6} (SEQ ID NO:7), Phe_{X-C4} (SEQ ID NO: 9), Phe_{X-D7} (SEQ ID NO:11), Phe_{X-A11} (SEQ ID NO:13), Phe_{X-B5} (SEQ ID NO:4), Phe_{X-D6} (SEQ ID NO:2), Phe_{X-A12} (SEQ ID NO:6), Phe_{X-B6} (SEQ ID NO:8), Phe_{X-C4} (SEQ ID NO:10), Phe_{X-D7} (SEQ ID NO:12), or Phe_{X-A11} (SEQ ID NO:12) and contacting the cell with 2,3,6F Phe.

12. The method of claim 11, wherein the synthetase is Phe_{X-B5} (SEQ ID NO:3), Phe_{X-D6} (SEQ ID NO:1), Phe_{X-B5} (SEQ ID NO:4), or Phe_{X-D6} (SEQ ID NO:2).

13. The method of claim 11 or 12, wherein the cell is a prokaryotic cell, and the synthetase is Phe_{X-B5} (SEQ ID NO:3), Phe_{X-D6} (SEQ ID NO:1), Phe_{X-A12} (SEQ ID NO: 5), Phe_{X-B6} (SEQ ID NO:7), Phe_{X-C4} (SEQ ID NO:9), Phe_{X-D7} (SEQ ID NO:11), or Phe_{X-A11} (SEQ ID NO:13).

14. The method of claim 13, wherein the prokaryotic cell is an E. coli cell.

15. The method of claim 11 or 12, wherein the cell is a eukaryotic cell, and the synthetase is Phe_{X-B5} (SEQ ID NO:4), Phe_{X-D6} (SEQ ID NO:2), Phe_{X-A12} (SEQ ID NO:6), Phe_{X-B6} (SEQ ID NO:8), Phe_{X-C4} (SEQ ID NO:10), Phe_{X-D7} (SEQ ID NO:12), or Phe_{X-A11} (SEQ ID NO:12).

16. The method of claim 15, wherein the eukaryotic cell is a mammalian cell.

17. A Pheₓ synthetase that is competent for site-specific encoding of fluorinated di-, tr-, tetra- and penta-fluoro phenylalanine analogs within protein in both bacterial and mammalian expression systems.

18. A Pyrrolysine-based aminoacyl-tRNA synthetase generated in E. coli.

19. The Pyrrolysine-based aminoacyl-tRNA synthetase of claim 18, wherein the Pyrrolysine-based aminoacyl-tRNA synthetase is selected from the group consisting of Phe_{X-B5} (SEQ ID NO:3), Phe_{X-D6} (SEQ ID NO:1), Phe_{X-A12} (SEQ ID NO:5), Phe_{X-B6} (SEQ ID NO:7), Phe_{X-C4} (SEQ ID NO:9), Phe_{X-D7} (SEQ ID NO:11), and Phe_{X-A11} (SEQ ID NO:13).

20. The Pyrrolysine-based aminoacyl-tRNA synthetase of claim 19, wherein the Pyrrolysine-based aminoacyl-tRNA synthetase is Phe_{X-B5} (SEQ ID NO:3).

21. The Pyrrolysine-based aminoacyl-tRNA synthetase of claim 19, wherein the Pyrrolysine-based aminoacyl-tRNA synthetase is Phe_{X-D6} (SEQ ID NO:1).

22. The Pyrrolysine-based aminoacyl-tRNA synthetase of claim 19, wherein the Pyrrolysine-based aminoacyl-tRNA synthetase is Phe_{X-A12} (SEQ ID NO:5).

23. The Pyrrolysine-based aminoacyl-tRNA synthetase of claim 19, wherein the Pyrrolysine-based aminoacyl-tRNA synthetase is Phe_{X-B6} (SEQ ID NO:7).

24. The Pyrrolysine-based aminoacyl-tRNA synthetase of claim 19, wherein the Pyrrolysine-based aminoacyl-tRNA synthetase is Phe_{X-C4} (SEQ ID NO:9).

25. The Pyrrolysine-based aminoacyl-tRNA synthetase of claim 19, wherein the Pyrrolysine-based aminoacyl-tRNA synthetase is Phe_{X-D7} (SEQ ID NO:11).

26. The Pyrrolysine-based aminoacyl-tRNA synthetase of claim 19, wherein the Pyrrolysine-based aminoacyl-tRNA synthetase is Phe_{X-A11} (SEQ ID NO:13).

27. A Pyrrolysine-based aminoacyl-tRNA synthetase generated in a mammalian cell.

28. The Pyrrolysine-based aminoacyl-tRNA synthetase of claim 27, wherein the mammalian cell is a human embryonic kidney (HEK) cell.

29. The Pyrrolysine-based aminoacyl-tRNA synthetase of claim 27 or 28, wherein the Pyrrolysine-based aminoacyl-tRNA synthetase is selected from the group consisting of Phe_{X-B5} (SEQ ID NO:4), Phe_{X-D6} (SEQ ID NO:2), Phe_{X-A12} (SEQ ID NO:6), Phe_{X-B6} (SEQ ID NO:8), Phe_{X-C4} (SEQ ID NO:10), Phe_{X-D7} (SEQ ID NO:12), and Phe_{X-A11} (SEQ ID NO:14).

30. The Pyrrolysine-based aminoacyl-tRNA synthetase of claim 29, wherein the Pyrrolysine-based aminoacyl-tRNA synthetase is Phe_{X-B5} (SEQ ID NO:4).

31. The Pyrrolysine-based aminoacyl-tRNA synthetase of claim 29, wherein the Pyrrolysine-based aminoacyl-tRNA synthetase is Phe_{X-D6} (SEQ ID NO:2).

32. The Pyrrolysine-based aminoacyl-tRNA synthetase of claim 29, wherein the Pyrrolysine-based aminoacyl-tRNA synthetase is Phe_{X-A12} (SEQ ID NO:6).

33. The Pyrrolysine-based aminoacyl-tRNA synthetase of claim 29, wherein the Pyrrolysine-based aminoacyl-tRNA synthetase is Phe_{X-B6} (SEQ ID NO:8).

34. The Pyrrolysine-based aminoacyl-tRNA synthetase of claim 29, wherein the Pyrrolysine-based aminoacyl-tRNA synthetase is Phe_{X-C4} (SEQ ID NO:10).

35. The Pyrrolysine-based aminoacyl-tRNA synthetase of claim 29, wherein the Pyrrolysine-based aminoacyl-tRNA synthetase is Phe_{X-D7} (SEQ ID NO:12).

36. The Pyrrolysine-based aminoacyl-tRNA synthetase of claim 29, wherein the Pyrrolysine-based aminoacyl-tRNA synthetase is Phe_{X-A11} (SEQ ID NO:14).

37. A vector comprising a plasmid and a nucleic acid encoding synthetase of any one of claims 20-26 and 30-36.

38. The vector of claim 37, wherein the plasmid is pAcBac1.

39. A method of expressing sfGFP_N150TAG_HIS in a cell by contacting the cell with the Pyrrolysine-based aminoacyl-tRNA synthetase of any one of claims 20-26 and 30-36.

40. The method of claim 39, wherein the cell is a prokaryotic cell.

41. The method of claim 40, wherein the prokaryotic cell is an E. coli cell.

42. The method of claim 40, wherein the synthetase is Phe_{X-B5} (SEQ ID NO:3) or Phe_{X-D6} (SEQ ID NO:1).

43. The method of claim 39, wherein the cell is a eukaryotic cell.

44. The method of claim 43, wherein the eukaryotic cell is mammalian cell.

45. The method of claim 43, wherein the synthetase is Phe_{X-B5} (SEQ ID NO:4) or Phe_{X-D6} (SEQ ID NO:2).


SEQUENCE LISTING (20 sequences)

SEQ ID NO: 1 - protein, 419 aa, synthetic construct
MDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLYNYLRKLDRILPGPIKIFEVGPCYRKESDGKEHLEEFTMVAFMQMGSGCTRENLEALIKEFLDYLEIDFEIVGDSCMVYGDTLDIMHGDLELSSAGVGPVSLDREWGIDKPTIGAGFGLERLLKVMHGFKNIKRASRSESYYNGISTNL

SEQ ID NO: 2 - protein, 444 aa, synthetic construct
MACPVPLQLPPLERLTLDDYKDDDDKDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLYNYLRKLDRILPGPIKIFEVGPCYRKESDGKEHLEEFTMVAFMQMGSGCTRENLEALIKEFLDYLEIDFEIVGDSCMVYGDTLDIMHGDLELSSAGVGPVSLDREWGIDKPTIGAGFGLERLLKVMHGFKNIKRASRSESYYNGISTNL

SEQ ID NO: 3 - protein, 419 aa, synthetic construct
MDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLYNYLRKLDRILPGPIKIFEVGPCYRKESDGKEHLEEFTMVGFAQMGSGCTRENLEALIKEFLDYLEIDFEIVGDSCMVYGDTLDIMHGDLELSSAVVGPVSLDREWGIDKPWIGAGFGLERLLKVMHGFKNIKRASRSESYYNGISTNL

SEQ ID NO: 4 - protein, 444 aa, synthetic construct
MACPVPLQLPPLERLTLDDYKDDDDKDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLYNYLRKLDRILPGPIKIFEVGPCYRKESDGKEHLEEFTMVGFAQMGSGCTRENLEALIKEFLDYLEIDFEIVGDSCMVYGDTLDIMHGDLELSSAVVGPVSLDREWGIDKPWIGAGFGLERLLKVMHGFKNIKRASRSESYYNGISTNL

SEQ ID NO: 5 - protein, 419 aa, synthetic construct
MDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLYNYLRKLDRILPGPIKIFEVGPCYRKESDGKEHLEEFTMVGFCQMGSGCTRENLEALIKEFLDYLEIDFEIVGDSCMVYGDTLDIMHGDLELSSAAVGPVSLDREWGIDKPWIGAGFGLERLLKVMHGFKNIKRASRSESYYNGISTNL

SEQ ID NO: 6 - protein, 444 aa, synthetic construct
MACPVPLQLPPLERLTLDDYKDDDDKDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLYNYLRKLDRILPGPIKIFEVGPCYRKESDGKEHLEEFTMVGFCQMGSGCTRENLEALIKEFLDYLEIDFEIVGDSCMVYGDTLDIMHGDLELSSAAVGPVSLDREWGIDKPWIGAGFGLERLLKVMHGFKNIKRASRSESYYNGISTNL

SEQ ID NO: 7 - protein, 419 aa, synthetic construct; identical to SEQ ID NO: 1

SEQ ID NO: 8 - protein, 444 aa, synthetic construct; identical to SEQ ID NO: 2

SEQ ID NO: 9 - protein, 419 aa, synthetic construct
MDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLYNYLRKLDRILPGPIKIFEVGPCYRKESDGKEHLEEFTMVQFSQMGSGCTRENLEALIKEFLDYLEIDFEIVGDSCMVYGDTLDIMHGDLELSSAGVGPVSLDREWGIDKPTIGAGFGLERLLKVMHGFKNIKRASRSESYYNGISTNL

SEQ ID NO: 10 - protein, 444 aa, synthetic construct
MACPVPLQLPPLERLTLDDYKDDDDKDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLYNYLRKLDRILPGPIKIFEVGPCYRKESDGKEHLEEFTMVQFSQMGSGCTRENLEALIKEFLDYLEIDFEIVGDSCMVYGDTLDIMHGDLELSSAGVGPVSLDREWGIDKPTIGAGFGLERLLKVMHGFKNIKRASRSESYYNGISTNL

SEQ ID NO: 11 - protein, 419 aa, synthetic construct
MDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIRSPILIPAEYVERMGINNDTELSKQIFRVDKNLCLRPMLAPTLYNYLRKLDRILPGPIKIFEVGPCYRKESDGKEHLEEFTMVAFMQMGSGCTRENLEALIKEFLDYLEIDFEIVGDSCMVYGDTLDIMHGDLELSSAGVGPVSLDREWGIDKPTIGAGFGLERLLKVMHGFKNIKRASRSESYYNGISTNL

SEQ ID NO: 12 - protein, 444 aa, synthetic construct
MACPVPLQLPPLERLTLDDYKDDDDKDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIRSPILIPAEYVERMGINNDTELSKQIFRVDKNLCLRPMLAPTLYNYLRKLDRILPGPIKIFEVGPCYRKESDGKEHLEEFTMVAFMQMGSGCTRENLEALIKEFLDYLEIDFEIVGDSCMVYGDTLDIMHGDLELSSAGVGPVSLDREWGIDKPTIGAGFGLERLLKVMHGFKNIKRASRSESYYNGISTNL

SEQ ID NO: 13 - protein, 419 aa, synthetic construct
MDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLYNYLRKLDRILPGPIKIFEVGPCYRKESDGKEHLEEFTMVAFMQMGSGCTRENLEALIKEFLDYLEIDFEIVGDSCMVYGDTLDIMHGDLELSSANVGPVSLDREWGIDKPTIGAGFGLERLLKVMHGFKNIKRASRSESYYNGISTNL

SEQ ID NO: 14 - protein, 444 aa, synthetic construct
MACPVPLQLPPLERLTLDDYKDDDDKDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLYNYLRKLDRILPGPIKIFEVGPCYRKESDGKEHLEEFTMVAFMQMGSGCTRENLEALIKEFLDYLEIDFEIVGDSCMVYGDTLDIMHGDLELSSANVGPVSLDREWGIDKPTIGAGFGLERLLKVMHGFKNIKRASRSESYYNGISTNL

SEQ ID NO: 15 - protein, 249 aa, synthetic construct
MVSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHTAGVYITADKQKNGIKANFKIRHNVEDGSVQLADHYQQNTPIGDGPVLLPDNHYLSTQSVLSKDPNEKRDHMVLLEFVTAAGITHGMDELYKGSHHHHHH

SEQ ID NO: 16 - protein, 263 aa, synthetic construct
MVSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHTAGVYITADKQKNGIKANFKIRHNVEDGSVQLADHYQQNTPIGDGPVLLPDNHYLSTQSVLSKDPNEKRDHMVLLEFVTAAGITHGMDELYKGSGKPIPNPLLGLDSTHHHHHH

SEQ ID NO: 17 - RNA, 72 nt, unidentified
  source 1..72: mol_type = tRNA; note = Methanogenic archaea
ggaaacctgatcatgtagatcgaacggactctaaatccgttcagccgggttagattcccggggtttccgcca

SEQ ID NO: 18 - RNA, 73 nt, synthetic construct
  source 1..73: mol_type = other RNA
sggaaacctgatcatgtagatcgaacggactctaaatccgttcagccgggttagattcccggggtttccgcca

SEQ ID NO: 19 - protein, 8 aa, synthetic construct
KLGGQDIF

SEQ ID NO: 20 - protein, 7 aa, synthetic construct
MTEEQKK